(19) United States
(12) Patent Application Publication
(10) Pub. No.: US 2023/0177370 A1
(43) Pub. Date: Jun. 8, 2023

(54) METHODS AND SYSTEMS FOR PRECISION ONCOLOGY USING A MULTILEVEL BAYESIAN MODEL

(71) Applicant: xCures, Inc., Oakland, CA (US)

(72) Inventors: Asher Wasserman, Santa Cruz, CA (US); Mark Shapiro, Durham, NC (US); Jeffrey C. Shrager, Palo Alto, CA (US); Glenn A. Kramer, San Francisco, CA (US)

(21) Appl. No.: 18/074,659

(22) Filed: Dec. 5, 2022

Related U.S. Application Data
(63) Continuation of application No. PCT/US2021/035759, filed on Jun. 3, 2021.
(60) Provisional application No. 63/034,578, filed on Jun. 4, 2020.
(60) Provisional application No. 63/094,478, filed on Oct. 21, 2020.

(51) Int. Cl. G06N 7/01; G06N 20/00

(52) U.S. Cl. CPC G06N 7/01; G06N 20/00

(57) ABSTRACT

In an aspect, the present disclosure provides a system comprising a computer processor and a storage device having instructions stored thereon that are operable, when executed by the computer processor, to cause the computer processor to: (i) receive clinical data of a subject and a set of treatment options for a disease or disorder of the subject, wherein the set of treatment options corresponds to clinical outcomes having future uncertainty; (ii) access a prediction module comprising a trained machine learning model that determines probabilistic predictions of clinical outcomes of the set of treatment options based at least in part on clinical data of test subjects; and (iii) apply the prediction module to at least the clinical data of the subject to determine probabilistic predictions of clinical outcomes of the set of treatment options.

### CROSS-REFERENCE

This application is a continuation of International Application No. PCT/US2021/035759, filed Jun. 3, 2021, which claims the benefit of U.S. Provisional Pat. Application No. 63/034,578, filed Jun. 4, 2020, and U.S. Provisional Pat. Application No. 63/094,478, filed Oct. 21, 2020, each of which is incorporated by reference herein in its entirety.

### BACKGROUND

Physicians engaged in precision oncology may integrate an overwhelming amount of information from publications and from their own experience. For example, as of the end of 2019, PubMed reports 19,748 publications matching the term “breast cancer” in the past year alone, and the same search for open, recruiting studies in ClinicalTrials.gov returns 1,937 studies. Therefore, practitioners, such as oncologists, may face challenges in reading all these materials, determining which may be most relevant, and synthesizing the whole of the data into relevant predictions for patient outcomes.

### SUMMARY

Oncologists fighting less common cancers may be potentially a worse situation; instead of being overwhelmed, they may have only a few relevant publications, and may have seen only a small number of similar cases. Here, successful prediction of patient outputs may depend on prior information gleaned from experts in similar, but not exactly the same, disease states.

Importantly, prediction may not be an exact science. Every patient may respond differently, due to a multitude of unknowns; it may be difficult or even impossible to fully model patients and their disease states, or the complete set of interactions between patients and their treatment regimens.

For some cancers, such as chronic myelogenous leukemia, the level of uncertainty may be relatively low; patients may almost universally receive tyrosine kinase receptor inhibitors, and the response characteristics may be relatively well-known. But for most cancers, and for many late-stage cancers, the number of unknown variables may far outnumber the number of known characteristics. In these cases, the sum of effects from the unknown variables may exceed the effects from known treatments. This may require probabilistic reasoning in order to devise an effective rational treatment strategy.

Thus, there remains a need for automated intelligent systems and methods that acquire and structure knowledge from a diverse array of sources, such as clinical trials, case series, individual patients cases and outcomes data, and expert opinions, such that such information may be used to predict, for a given patient, what the probable range of outcomes might be, over time, for a given treatment. Furthermore, such predictions may be explainable to a physician or scientist who queries the system for such a prediction; in contrast, a “black box” that provides answers without rationales may not instill confidence.

In light of the needs above, the present disclosure provides systems and methods for precision oncology using multilevel Bayesian models, which may effectively address challenges faced by physicians when treating patients with complex disease etiologies, such as cancer. Systems and methods of the present disclosure may be used to predict various measures of patient outcomes for particular patients under different treatment regimens. The systems and methods may be capable of learning from a diverse range of information sources, including individual patient outcomes observed outside of randomized trials (in other words, “real world evidence” or RWE) as well as other sources, such as expert surveys and summary statistics from clinical trials. The learning process may occur via a training module, which presents this data in a learning loop to a multilevel model module, which may be a combination of a Bayesian model and database.

Once the multilevel model module has been conditioned on such source data, it may be used in conjunction with a prediction module to predict outcomes for new patients under different treatment choices and provide a measure of the uncertainty of these predictions. These predictions may be probabilistic in nature, in that they represent a distribution of possible outcomes (e.g., in contrast to a single outcome).

A key advance may be that the multilevel model’s structure bears an understandable relationship to the domain, and to the types of inputs and outputs oncologists may expect. This structure may help users of systems and methods of the present disclosure to understand how the predictions and uncertainty therein may be derived, rather than treating the results as “black box” predictions. This level of explainability may be critical, for example, for certification of medical devices that rely on Artificial Intelligence and Machine Learning.

In an aspect, the present disclosure provides a system comprising a computer processor and a storage device having instructions stored thereon that are operable, when executed by the computer processor, to cause the computer processor to: (i) receive clinical data of a subject and a set of treatment options for a disease or disorder of the subject, wherein the set of treatment options corresponds to clinical outcomes having future uncertainty; (ii) access a prediction module comprising a trained machine learning model that determines probabilistic predictions of clinical outcomes of the set of treatment options based at least in part on clinical data of subjects; and (iii) apply the prediction module to at least the clinical data of the subject to determine probabilistic predictions of clinical outcomes of the set of treatment options for the disease or disorder of the subject.

In some embodiments, the clinical data is selected from somatic genetic mutations, germline genetic mutations, mutational burden, protein levels, transcriptome levels, metabolite levels, tumor size or staging, clinical symptoms, laboratory test results, and clinical history.

In some embodiments, the disease or disorder comprises cancer. In some embodiments, the subject has received a previous treatment for the cancer. In some embodiments, the subject has not received a previous treatment for the cancer.

In some embodiments, the cancer is selected from the group consisting of: Adrenal Gland Tumor, Ampulla of Vater Tumor, Biliary Tract Tumor, Bladder/Urinary Tract Tumor, Bone Tumor, Bowel Tumor, Breast Tumor, CNS/Brain Tumor, Cervix Tumor, Esophagus/Stomach Tumor, Eye Tumor, Head and Neck Tumor, Kidney Tumor, Liver Tumor, Lung Tumor, Lymphoid Tumor, Myeloid Tumor, Other Tumor, Ovary/Fallopian Tube Tumor, Pancreas Tumor, Penis Tumor, Peripheral Nervous System Tumor, Peritoneum Tumor, Pleura Tumor, Prostate Tumor, Skin Tumor, Soft Tissue Tumor, Testis Tumor, Thymus Tumor, Thyroid Tumor, Uterus Tumor, and Vulva/Vagina Tumor. In some embodiments, the cancer is selected from the group consisting of: Adrenal Gland Tumor, Ampulla of Vater Tumor, Biliary Tract Tumor, Bladder/Urinary Tract Tumor, Bone Tumor, Bowel Tumor, Breast Tumor, CNS/Brain Tumor, Cervix Tumor, Esophagus/Stomach Tumor, Eye Tumor, Head and Neck Tumor, Kidney Tumor, Liver Tumor, Lung Tumor, Lymphoid Tumor, Myeloid Tumor, Other Tumor, Ovary/Fallopian Tube Tumor, Pancreas Tumor, Penis Tumor, Peripheral Nervous System Tumor, Peritoneum Tumor, Pleura Tumor, Prostate Tumor, Skin Tumor, Soft Tissue Tumor, Testis Tumor, Thymus Tumor, Thyroid Tumor, Uterus Tumor, Vulva/Vagina Tumor, Adrenocortical Adenoma, Adrenocortical Carcinoma, Pheochromocytoma, Ampullary Carcinoma, Cholangiocarcinoma, Gallbladder Cancer, Intracholecystic Papillary Neoplasm, Intraductal Papillary Neoplasm of the Bile Duct, Bladder Adenocarcinoma, Bladder Squamous Cell Carcinoma, Bladder Urothelial Carcinoma, Inflammatory Myofibroblastic Bladder Tumor, Inverted Urothelial Papilloma, Mucosal Melanoma of the Urethra, Plasmacytoid/Signet Ring Cell Bladder Carcinoma, Sarcomatoid Carcinoma of the Urinary Bladder, Small Cell Bladder Cancer, Upper Tract Urothelial Carcinoma, Urachal Carcinoma, Urethral Cancer, Urothelial Papilloma, Adamantinoma, Chondroblastoma, Chondrosarcoma, Chordoma, Ewing Sarcoma, Giant Cell Tumor of Bone, Osteosarcoma, Anal Gland Adenocarcinoma, Anal Squamous Cell Carcinoma, Anorectal Mucosal Melanoma, Appendiceal Adenocarcinoma, Colorectal Adenocarcinoma, Gastrointestinal Neuroendocrine Tumors, Low-grade Appendiceal Mucinous Neoplasm, Medullary Carcinoma of the Colon, Small Bowel Cancer, Small Intestinal Carcinoma, Tubular Adenoma of the Colon, Adenomyoepithelioma of the Breast, Breast Ductal Carcinoma In Situ, Breast Fibroepithelial Neoplasms, Breast Lobular Carcinoma In Situ, Breast Neoplasm, NOS, Breast Sarcoma, Inflammatory Breast Cancer, Invasive Breast Carcinoma, Juvenile Secretory Carcinoma of the Breast, Metaplastic Breast Cancer, Choroid Plexus Tumor, Diffuse Glioma, Embryonal Tumor, Encapsulated Glioma, Ependymomal Tumor, Germ Cell Tumor, Brain, Meningothelial Tumor, Miscellaneous Brain Tumor, Miscellaneous Neuroepithelial Tumor, Pineal Tumor, Primary CNS Melanocytic Tumors, Sellar Tumor, Cervical Adenocarcinoma, Cervical Adenocarcinoma In Situ, Cervical Adenoid Basal Carcinoma, Cervical Adenoid Cystic Carcinoma, Cervical Adenosquamous Carcinoma, Cervical Leiomyosarcoma, Cervical Neuroendocrine Tumor, Cervical Rhabdomyosarcoma, Cervical Squamous Cell Carcinoma, Glassy Cell Carcinoma of the Cervix, Mixed Cervical Carcinoma, Small Cell Carcinoma of the Cervix, Villoglandular Adenocarcinoma of the Cervix, Esophageal Poorly Differentiated Carcinoma, Esophageal Squamous Cell Carcinoma, Esophagogastric Adenocarcinoma, Gastrointestinal Neuroendocrine Tumors of the Esophagus/Stomach, Mucosal Melanoma of the Esophagus, Smooth Muscle Neoplasm, NOS, Lacrimal Gland Tumor, Ocular Melanoma, Retinoblastoma, Head and Neck Carcinoma, Other, Head and Neck Mucosal Melanoma, Head and Neck Squamous Cell Carcinoma, Nasopharyngeal Carcinoma, Parathyroid Cancer, Salivary Carcinoma, Sialoblastoma, Clear Cell Sarcoma of Kidney, Renal Cell Carcinoma, Renal Neuroendocrine Tumor, Rhabdoid Cancer, Wilms’ Tumor, Fibrolamellar Carcinoma, Hepatoblastoma, Hepatocellular Adenoma, Hepatocellular Carcinoma, Hepatocellular Carcinoma plus Intrahepatic Cholangiocarcinoma, Liver Angiosarcoma, Malignant Nonepithelial Tumor of the Liver, Malignant Rhabdoid Tumor of the Liver, Undifferentiated Embryonal Sarcoma of the Liver, Combined Small Cell Lung Carcinoma, Inflammatory Myofibroblastic Lung Tumor, Lung Adenocarcinoma In Situ, Lung Neuroendocrine Tumor, Non-Small Cell Lung Cancer, Pleuropulmonary Blastoma, Pulmonary Lymphangiomyomatosis, Sarcomatoid Carcinoma of the Lung, Lymphoid Atypical, Lymphoid Benign, Lymphoid Neoplasm, Myeloid Atypical, Myeloid Benign, Myeloid Neoplasm, Adenocarcinoma In Situ, Cancer of Unknown Primary, Extra Gonadal Germ Cell Tumor, Mixed Cancer Types, Ovarian Cancer, Other, Ovarian Epithelial Tumor, Ovarian Germ Cell Tumor, Sex Cord Stromal Tumor, Acinar Cell Carcinoma of the Pancreas, Adenosquamous Carcinoma of the Pancreas, Cystic Tumor of the Pancreas, Pancreatic Adenocarcinoma, Pancreatic Neuroendocrine Tumor, Pancreatoblastoma, Solid Pseudopapillary Neoplasm of the Pancreas, Undifferentiated Carcinoma of the Pancreas, Penile Squamous Cell Carcinoma, Ganglioneuroblastoma, Ganglioneuroma, Nerve Sheath Tumor, Neuroblastoma, Peritoneal Mesothelioma, Peritoneal Serous Carcinoma, Pleural Mesothelioma, Basal Cell Carcinoma of Prostate, Prostate Adenocarcinoma, Prostate Neuroendocrine Carcinoma, Prostate Small Cell Carcinoma, Prostate Squamous Cell Carcinoma, Aggressive Digital Papillary Adenocarcinoma, Atypical Fibroxanthoma, Atypical Nevus, Basal Cell Carcinoma, Cutaneous Squamous Cell Carcinoma, Dermatofibroma, Dermatofibrosarcoma Protuberans, Desmoplastic Trichoepithelioma, Endocrine Mucin Producing Sweat Gland Carcinoma, Extramammary Paget Disease, Melanoma, Merkel Cell Carcinoma, Microcystic Adnexal Carcinoma, Porocarcinoma/Spiroadenocarcinoma, Poroma/Acrospiroma, Proliferating Pilar Cystic Tumor, Sebaceous Carcinoma, Skin Adnexal Carcinoma, Spiroma/Spiradenoma, Sweat Gland Adenocarcinoma, Sweat Gland Carcinoma/Apocrine Eccrine Carcinoma, Aggressive Angiomyxoma, Alveolar Soft Part Sarcoma, Angiomatoid Fibrous Histiocytoma, Angiosarcoma, Atypical Lipomatous Tumor, Clear Cell Sarcoma, Dendritic Cell Sarcoma, Desmoid/Aggressive Fibromatosis, Desmoplastic Small-Round-Cell Tumor, Epithelioid Hemangioendothelioma, Epithelioid Sarcoma, Ewing Sarcoma of Soft Tissue, Fibrosarcoma, Gastrointestinal Stromal Tumor, Glomangiosarcoma, Hemangioma, Infantile Fibrosarcoma, Inflammatory Myofibroblastic Tumor, Intimal Sarcoma, Leiomyoma, Leiomyosarcoma, Liposarcoma, Low-Grade Fibromyxoid Sarcoma, Malignant Glomus Tumor, Myofibroma, Myofibromatosis, Myopericytoma, Myxofibrosarcoma, Myxoma, Paraganglioma, Perivascular Epithelioid Cell Tumor, Pseudomyogenic Hemangioendothelioma, Radiation-Associated Sarcoma, Rhabdomyosarcoma, Round Cell Sarcoma, NOS, Sarcoma, NOS, Soft Tissue Myoepithelial Carcinoma, Solitary Fibrous Tumor/Hemangiopericytoma, Synovial Sarcoma, Tenosynovial Giant Cell Tumor Diffuse Type, Undifferentiated Pleomorphic Sarcoma/Malignant Fibrous Histiocytoma/High-Grade Spindle Cell Sarcoma, Non-Seminomatous Germ Cell Tumor, Seminoma, Sex Cord Stromal Tumor, Testicular Lymphoma, Testicular Mesothelioma, Thymic Epithelial Tumor, Thymic Neuroendocrine Tumor, Anaplastic Thyroid Cancer, Hurthle Cell Thyroid Cancer, Hyalinizing Trabecular Adenoma of the Thyroid, Medullary Thyroid Cancer, Oncocytic Adenoma of the Thyroid, Poorly Differentiated Thyroid Cancer, Well-Differentiated Thyroid Cancer, Endometrial Carcinoma, Gestational Trophoblastic Disease, Other Uterine Tumor, Uterine Sarcoma/Mesenchymal, Germ Cell Tumor of the Vulva, Mucinous Adenocarcinoma of the Vulva/Vagina, Mucosal Melanoma of the Vulva/Vagina, Poorly Differentiated Vaginal Carcinoma, Squamous Cell Carcinoma of the Vulva/Vagina, and Vaginal Adenocarcinoma.

In some embodiments, (iii) comprises applying the prediction module to at least treatment features of the set of treatment options to determine the probabilistic predictions of the clinical outcomes of the set of treatment options. In some embodiments, the treatment features comprise attributes of a surgical intervention, a drug intervention, a targeted intervention, a hormonal therapy intervention, a radiotherapy intervention, or an immunotherapy intervention. In some embodiments, the treatment features comprise the attributes of the drug intervention, wherein the attributes of the drug intervention comprise a chemical structure or a biological target of the drug intervention.

In some embodiments, (iii) comprises applying the prediction module to at least interaction terms between the clinical data of the subject and the treatment features of the set of treatment options to determine the probabilistic predictions of the clinical outcomes of the set of treatment options.

In some embodiments, the clinical outcomes having future uncertainty comprise a change in tumor size, a change in patient functional status, a time-to-disease progression, a time-to-treatment failure, overall survival, or progression-free survival. In some embodiments, the clinical outcomes having future uncertainty comprise the change in tumor size, as indicated by cross section or volume. In some embodiments, the clinical outcomes having future uncertainty comprise the change in patient functional status, as indicated by ECOG, Karnofsky, or Lansky scores.

In some embodiments, the probabilistic predictions of clinical outcomes of the set of treatment options comprise statistical distributions of the clinical outcomes of the set of treatment options. In some embodiments, (iii) further comprises determining a statistical parameter of the statistical distributions of the clinical outcomes of the set of treatment options. In some embodiments, the statistical parameter is selected from the group consisting of a median, a mean, a mode, a variance, a standard deviation, a quantile, a measure of central tendency, a measure of variance, a range, a minimum, a maximum, an interquartile range, a frequency, a percentile, a shape parameter, a scale parameter, and a rate parameter. In some embodiments, the statistical distributions of the clinical outcomes of the set of treatment options comprise a parametric distribution selected from the group consisting of a Weibull distribution, a log logistic distribution, or a log normal distribution, a Gaussian distribution, a Gamma distribution, and a Poisson distribution.

In some embodiments, the probabilistic predictions of clinical outcomes of the set of treatment options are explainable based on performing a query of the probabilistic predictions.

In some embodiments, the instructions are operable, when executed by the computer processor, to cause the computer processor to further apply a training module that trains the trained machine learning model. In some embodiments, the trained machine learning model is trained using a plurality of disparate data sources. In some embodiments, the training module aggregates datasets from the plurality of disparate sources, wherein the datasets are persisted in a plurality of data stores, and trains the trained machine learning model using the aggregated datasets. In some embodiments, the plurality of disparate sources is selected from the group consisting of clinical trials, case series, individual patient cases and outcomes data, and expert opinions.

In some embodiments, the training module updates the trained machine learning model using the probabilistic predictions of the clinical outcomes of the set of treatment options generated in (iii). In some embodiments, updating is performed using a Bayesian update or a maximum likelihood algorithm.

In some embodiments, the trained machine learning model is selected from the group consisting of a Bayesian model, a support vector machine (SVM), a linear regression, a logistic regression, a random forest, and a neural network. In some embodiments, the trained machine learning model comprises a multilevel statistical model that accounts for variation at a plurality of distinct levels of analysis. In some embodiments, the multilevel statistical model accounts for correlation of subject-level effects across the plurality of distinct levels of analysis.

In some embodiments, the multilevel statistical model comprises a generalized linear model. In some embodiments, the generalized linear model comprises use of the expression:

$$η = X ⋅ β + Z ⋅ u$$

, wherein η is a linear response, X is a vector of predictors for treatment effects fixed across subjects, β is a vector of fixed effects, Z is a vector of predictors for subject-level treatment effects, and u is a vector of subject-level effects. In some embodiments, the generalized linear model comprises use of the expression: y = g⁻¹(η), wherein η is a linear response, g is an appropriately chosen link function from observed data to the linear response, and y is an outcome variable of interest.

In some embodiments, (iii) comprises applying a plurality of iterations of the prediction module to determine the probabilistic predictions of the clinical outcomes of the set of treatment options.

In some embodiments, the instructions are operable, when executed by the computer processor, to cause the computer processor to further use a parsing module to identify relevant features of the clinical data of the subject, the set of treatment options, and/or interaction terms between the clinical data of the subject and the treatment features of the set of treatment options. In some embodiments, the parsing module identifies relevant features by matching against a feature library.

In some embodiments, the instructions are operable, when executed by the computer processor, to cause the computer processor to further generate an electronic report comprising the probabilistic predictions of clinical outcomes of the set of treatment options. In some embodiments, the electronic report is used to select a treatment option from among the set of treatment options based at least in part on the probabilistic predictions of clinical outcomes of the set of treatment options. In some embodiments, the selected treatment option is administered to the subject. In some embodiments, the prediction module is further applied to outcome data of the subject that is obtained subsequent to administering the selected treatment option to the subject, to determine updated probabilistic predictions of the clinical outcomes of the set of treatment options.

In another aspect, the present disclosure provides a computer-implemented method comprising: (i) receiving clinical data of a subject and a set of treatment options for a disease or disorder of the subject, wherein the set of treatment options corresponds to clinical outcomes having future uncertainty; (ii) accessing a prediction module comprising a trained machine learning model that determines probabilistic predictions of clinical outcomes of the set of treatment options based at least in part on clinical data of test subjects; and (iii) applying the prediction module to at least the clinical data of the subject to determine probabilistic predictions of clinical outcomes of the set of treatment options for the disease or disorder of the subject.

In some embodiments, the clinical data is selected from somatic genetic mutations, germline genetic mutations, mutational burden, protein levels, transcriptome levels, metabolite levels, tumor size or staging, clinical symptoms, laboratory test results, and clinical history.

In some embodiments, the disease or disorder comprises cancer. In some embodiments, the subject has received a previous treatment for the cancer. In some embodiments, the subject has not received a previous treatment for the cancer.

In some embodiments, the cancer is selected from the group consisting of: Adrenal Gland Tumor, Ampulla of Vater Tumor, Biliary Tract Tumor, Bladder/Urinary Tract Tumor, Bone Tumor, Bowel Tumor, Breast Tumor, CNS/Brain Tumor, Cervix Tumor, Esophagus/Stomach Tumor, Eye Tumor, Head and Neck Tumor, Kidney Tumor, Liver Tumor, Lung Tumor, Lymphoid Tumor, Myeloid Tumor, Other Tumor, Ovary/Fallopian Tube Tumor, Pancreas Tumor, Penis Tumor, Peripheral Nervous System Tumor, Peritoneum Tumor, Pleura Tumor, Prostate Tumor, Skin Tumor, Soft Tissue Tumor, Testis Tumor, Thymus Tumor, Thyroid Tumor, Uterus Tumor, and Vulva/Vagina Tumor. In some embodiments, the cancer is selected from the group consisting of: Adrenal Gland Tumor, Ampulla of Vater Tumor, Biliary Tract Tumor, Bladder/Urinary Tract Tumor, Bone Tumor, Bowel Tumor, Breast Tumor, CNS/Brain Tumor, Cervix Tumor, Esophagus/Stomach Tumor, Eye Tumor, Head and Neck Tumor, Kidney Tumor, Liver Tumor, Lung Tumor, Lymphoid Tumor, Myeloid Tumor, Other Tumor, Ovary/Fallopian Tube Tumor, Pancreas Tumor, Penis Tumor, Peripheral Nervous System Tumor, Peritoneum Tumor, Pleura Tumor, Prostate Tumor, Skin Tumor, Soft Tissue Tumor, Testis Tumor, Thymus Tumor, Thyroid Tumor, Uterus Tumor, Vulva/Vagina Tumor, Adrenocortical Adenoma, Adrenocortical Carcinoma, Pheochromocytoma, Ampullary Carcinoma, Cholangiocarcinoma, Gallbladder Cancer, Intracholecystic Papillary Neoplasm, Intraductal Papillary Neoplasm of the Bile Duct, Bladder Adenocarcinoma, Bladder Squamous Cell Carcinoma, Bladder Urothelial Carcinoma, Inflammatory Myofibroblastic Bladder Tumor, Inverted Urothelial Papilloma, Mucosal Melanoma of the Urethra, Plasmacytoid/Signet Ring Cell Bladder Carcinoma, Sarcomatoid Carcinoma of the Urinary Bladder, Small Cell Bladder Cancer, Upper Tract Urothelial Carcinoma, Urachal Carcinoma, Urethral Cancer, Urothelial Papilloma, Adamantinoma, Chondroblastoma, Chondrosarcoma, Chordoma, Ewing Sarcoma, Giant Cell Tumor of Bone, Osteosarcoma, Anal Gland Adenocarcinoma, Anal Squamous Cell Carcinoma, Anorectal Mucosal Melanoma, Appendiceal Adenocarcinoma, Colorectal Adenocarcinoma, Gastrointestinal Neuroendocrine Tumors, Low-grade Appendiceal Mucinous Neoplasm, Medullary Carcinoma of the Colon, Small Bowel Cancer, Small Intestinal Carcinoma, Tubular Adenoma of the Colon, Adenomyoepithelioma of the Breast, Breast Ductal Carcinoma In Situ, Breast Fibroepithelial Neoplasms, Breast Lobular Carcinoma In Situ, Breast Neoplasm, NOS, Breast Sarcoma, Inflammatory Breast Cancer, Invasive Breast Carcinoma, Juvenile Secretory Carcinoma of the Breast, Metaplastic Breast Cancer, Choroid Plexus Tumor, Diffuse Glioma, Embryonal Tumor, Encapsulated Glioma, Ependymomal Tumor, Germ Cell Tumor, Brain, Meningothelial Tumor, Miscellaneous Brain Tumor, Miscellaneous Neuroepithelial Tumor, Pineal Tumor, Primary CNS Melanocytic Tumors, Sellar Tumor, Cervical Adenocarcinoma, Cervical Adenocarcinoma In Situ, Cervical Adenoid Basal Carcinoma, Cervical Adenoid Cystic Carcinoma, Cervical Adenosquamous Carcinoma, Cervical Leiomyosarcoma, Cervical Neuroendocrine Tumor, Cervical Rhabdomyosarcoma, Cervical Squamous Cell Carcinoma, Glassy Cell Carcinoma of the Cervix, Mixed Cervical Carcinoma, Small Cell Carcinoma of the Cervix, Villoglandular Adenocarcinoma of the Cervix, Esophageal Poorly Differentiated Carcinoma, Esophageal Squamous Cell Carcinoma, Esophagogastric Adenocarcinoma, Gastrointestinal Neuroendocrine Tumors of the Esophagus/Stomach, Mucosal Melanoma of the Esophagus, Smooth Muscle Neoplasm, NOS, Lacrimal Gland Tumor, Ocular Melanoma, Retinoblastoma, Head and Neck Carcinoma, Other, Head and Neck Mucosal Melanoma, Head and Neck Squamous Cell Carcinoma, Nasopharyngeal Carcinoma, Parathyroid Cancer, Salivary Carcinoma, Sialoblastoma, Clear Cell Sarcoma of Kidney, Renal Cell Carcinoma, Renal Neuroendocrine Tumor, Rhabdoid Cancer, Wilms’ Tumor, Fibrolamellar Carcinoma, Hepatoblastoma, Hepatocellular Adenoma, Hepatocellular Carcinoma, Hepatocellular Carcinoma plus Intrahepatic Cholangiocarcinoma, Liver Angiosarcoma, Malignant Nonepithelial Tumor of the Liver, Malignant Rhabdoid Tumor of the Liver, Undifferentiated Embryonal Sarcoma of the Liver, Combined Small Cell Lung Carcinoma, Inflammatory Myofibroblastic Lung Tumor, Lung Adenocarcinoma In Situ, Lung Neuroendocrine Tumor, Non-Small Cell Lung Cancer, Pleuropulmonary Blastoma, Pulmonary Lymphangiomyomatosis, Sarcomatoid Carcinoma of the Lung, Lymphoid Atypical, Lymphoid Benign, Lymphoid Neoplasm, Myeloid Atypical, Myeloid Benign, Myeloid Neoplasm, Adenocarcinoma In Situ, Cancer of Unknown Primary, Extra Gonadal Germ Cell Tumor, Mixed Cancer Types, Ovarian Cancer, Other, Ovarian Epithelial Tumor, Ovarian Germ Cell Tumor, Sex Cord Stromal Tumor, Acinar Cell Carcinoma of the Pancreas, Adenosquamous Carcinoma of the Pancreas, Cystic Tumor of the Pancreas, Pancreatic Adenocarcinoma, Pancreatic Neuroendocrine Tumor, Pancreatoblastoma, Solid Pseudopapillary Neoplasm of the Pancreas, Undifferentiated Carcinoma of the Pancreas, Penile Squamous Cell Carcinoma, Ganglioneuroblastoma, Ganglioneuroma, Nerve Sheath Tumor, Neuroblastoma, Peritoneal Mesothelioma, Peritoneal Serous Carcinoma, Pleural Mesothelioma, Basal Cell Carcinoma of Prostate, Prostate Adenocarcinoma, Prostate Neuroendocrine Carcinoma, Prostate Small Cell Carcinoma, Prostate Squamous Cell Carcinoma, Aggressive Digital Papillary Adenocarcinoma, Atypical Fibroxanthoma, Atypical Nevus, Basal Cell Carcinoma, Cutaneous Squamous Cell Carcinoma, Dermatofibroma, Dermatofibrosarcoma Protuberans, Desmoplastic Trichoepithelioma, Endocrine Mucin Producing Sweat Gland Carcinoma, Extramammary Paget Disease, Melanoma, Merkel Cell Carcinoma, Microcystic Adnexal Carcinoma, Porocarcinoma/Spiroadenocarcinoma, Poroma/Acrospiroma, Proliferating Pilar Cystic Tumor, Sebaceous Carcinoma, Skin Adnexal Carcinoma, Spiroma/Spiradenoma, Sweat Gland Adenocarcinoma, Sweat Gland Carcinoma/Apocrine Eccrine Carcinoma, Aggressive Angiomyxoma, Alveolar Soft Part Sarcoma, Angiomatoid Fibrous Histiocytoma, Angiosarcoma, Atypical Lipomatous Tumor, Clear Cell Sarcoma, Dendritic Cell Sarcoma, Desmoid/Aggressive Fibromatosis, Desmoplastic Small-Round-Cell Tumor, Epithelioid Hemangioendothelioma, Epithelioid Sarcoma, Ewing Sarcoma of Soft Tissue, Fibrosarcoma, Gastrointestinal Stromal Tumor, Glomangiosarcoma, Hemangioma, Infantile Fibrosarcoma, Inflammatory Myofibroblastic Tumor, Intimal Sarcoma, Leiomyoma, Leiomyosarcoma, Liposarcoma, Low-Grade Fibromyxoid Sarcoma, Malignant Glomus Tumor, Myofibroma, Myofibromatosis, Myopericytoma, Myxofibrosarcoma, Myxoma, Paraganglioma, Perivascular Epithelioid Cell Tumor, Pseudomyogenic Hemangioendothelioma, Radiation-Associated Sarcoma, Rhabdomyosarcoma, Round Cell Sarcoma, NOS, Sarcoma, NOS, Soft Tissue Myoepithelial Carcinoma, Solitary Fibrous Tumor/Hemangiopericytoma, Synovial Sarcoma, Tenosynovial Giant Cell Tumor Diffuse Type, Undifferentiated Pleomorphic Sarcoma/Malignant Fibrous Histiocytoma/High-Grade Spindle Cell Sarcoma, Non-Seminomatous Germ Cell Tumor, Seminoma, Sex Cord Stromal Tumor, Testicular Lymphoma, Testicular Mesothelioma, Thymic Epithelial Tumor, Thymic Neuroendocrine Tumor, Anaplastic Thyroid Cancer, Hurthle Cell Thyroid Cancer, Hyalinizing Trabecular Adenoma of the Thyroid, Medullary Thyroid Cancer, Oncocytic Adenoma of the Thyroid, Poorly Differentiated Thyroid Cancer, Well-Differentiated Thyroid Cancer, Endometrial Carcinoma, Gestational Trophoblastic Disease, Other Uterine Tumor, Uterine Sarcoma/Mesenchymal, Germ Cell Tumor of the Vulva, Mucinous Adenocarcinoma of the Vulva/Vagina, Mucosal Melanoma of the Vulva/Vagina, Poorly Differentiated Vaginal Carcinoma, Squamous Cell Carcinoma of the Vulva/Vagina, and Vaginal Adenocarcinoma.

In some embodiments, (iii) comprises applying the prediction module to at least treatment features of the set of treatment options to determine the probabilistic predictions of the clinical outcomes of the set of treatment options. In some embodiments, the treatment features comprise attributes of a surgical intervention, a drug intervention, a targeted intervention, a hormonal therapy intervention, a radiotherapy intervention, or an immunotherapy intervention. In some embodiments, the treatment features comprise the attributes of the drug intervention, wherein the attributes of the drug intervention comprise a chemical structure or a biological target of the drug intervention.

In some embodiments, (iii) comprises applying the prediction module to at least interaction terms between the clinical data of the subject and the treatment features of the set of treatment options to determine the probabilistic predictions of the clinical outcomes of the set of treatment options.

In some embodiments, the clinical outcomes having future uncertainty comprise a change in tumor size, a change in patient functional status, a time-to-disease progression, a time-to-treatment failure, overall survival, or progression-free survival. In some embodiments, the clinical outcomes having future uncertainty comprise the change in tumor size, as indicated by cross section or volume. In some embodiments, the clinical outcomes having future uncertainty comprise the change in patient functional status, as indicated by ECOG, Karnofsky, or Lansky scores.

In some embodiments, the probabilistic predictions of clinical outcomes of the set of treatment options comprise statistical distributions of the clinical outcomes of the set of treatment options. In some embodiments, (iii) further comprises determining a statistical parameter of the statistical distributions of the clinical outcomes of the set of treatment options. In some embodiments, the statistical parameter is selected from the group consisting of a median, a mean, a mode, a variance, a standard deviation, a quantile, a measure of central tendency, a measure of variance, a range, a minimum, a maximum, an interquartile range, a frequency, a percentile, a shape parameter, a scale parameter, and a rate parameter. In some embodiments, the statistical distributions of the clinical outcomes of the set of treatment options comprise a parametric distribution selected from the group consisting of a Weibull distribution, a log logistic distribution, or a log normal distribution, a Gaussian distribution, a Gamma distribution, and a Poisson distribution.

In some embodiments, the probabilistic predictions of clinical outcomes of the set of treatment options are explainable based on performing a query of the probabilistic predictions.

In some embodiments, the method further comprises applying a training module that trains the trained machine learning model. In some embodiments, the trained machine learning model is trained using a plurality of disparate data sources. In some embodiments, the training module aggregates datasets from the plurality of disparate sources, wherein the datasets are persisted in a plurality of data stores, and trains the trained machine learning model using the aggregated datasets. In some embodiments, the plurality of disparate sources is selected from the group consisting of clinical trials, case series, individual patient cases and outcomes data, and expert opinions.

In some embodiments, the training module updates the trained machine learning model using the probabilistic predictions of the clinical outcomes of the set of treatment options generated in (iii). In some embodiments, updating is performed using a Bayesian update or a maximum likelihood algorithm.

In some embodiments, the trained machine learning model is selected from the group consisting of a Bayesian model, a support vector machine (SVM), a linear regression, a logistic regression, a random forest, and a neural network. In some embodiments, the trained machine learning model comprises a multilevel statistical model that accounts for variation at a plurality of distinct levels of analysis. In some embodiments, the multilevel statistical model accounts for correlation of subject-level effects across the plurality of distinct levels of analysis.

In some embodiments, the multilevel statistical model comprises a generalized linear model. In some embodiments, the generalized linear model comprises use of the expression:

$$η = X ⋅ β + Z ⋅ u$$

, wherein η is a linear response, X is a vector of predictors for treatment effects fixed across subjects, β is a vector of fixed effects, Z is a vector of predictors for subject-level treatment effects, and u is a vector of subject-level effects. In some embodiments, the generalized linear model comprises use of the expression: y = g⁻¹(η), wherein η is a linear response, g is an appropriately chosen link function from observed data to the linear response, and y is an outcome variable of interest.

In some embodiments, (iii) comprises applying a plurality of iterations of the prediction module to determine the probabilistic predictions of the clinical outcomes of the set of treatment options.

In some embodiments, the method further comprises using a parsing module to identify relevant features of the clinical data of the subject, the set of treatment options, and/or interaction terms between the clinical data of the subject and the treatment features of the set of treatment options. In some embodiments, the parsing module identifies relevant features by matching against a feature library.

In some embodiments, the method further comprises generating an electronic report comprising the probabilistic predictions of clinical outcomes of the set of treatment options. In some embodiments, the electronic report is used to select a treatment option from among the set of treatment options based at least in part on the probabilistic predictions of clinical outcomes of the set of treatment options. In some embodiments, the selected treatment option is administered to the subject. In some embodiments, the method further comprises applying the prediction module to outcome data of the subject that is obtained subsequent to administering the selected treatment option to the subject, to determine updated probabilistic predictions of the clinical outcomes of the set of treatment options.

In another aspect, the present disclosure provides a non-transitory computer storage medium storing instructions that are operable, when executed by computer processors, to implement a method comprising: (i) receiving clinical data of a subject and a set of treatment options for a disease or disorder of the subject, wherein the set of treatment options corresponds to clinical outcomes having future uncertainty; (ii) accessing a prediction module comprising a trained machine learning model that determines probabilistic predictions of clinical outcomes of the set of treatment options based at least in part on clinical data of test subjects; and (iii) applying the prediction module to at least the clinical data of the subject to determine probabilistic predictions of clinical outcomes of the set of treatment options for the disease or disorder of the subject.

In some embodiments, the clinical data is selected from somatic genetic mutations, germline genetic mutations, mutational burden, protein levels, transcriptome levels, metabolite levels, tumor size or staging, clinical symptoms, laboratory test results, and clinical history.

In some embodiments, the disease or disorder comprises cancer. In some embodiments, the subject has received a previous treatment for the cancer. In some embodiments, the subject has not received a previous treatment for the cancer.

In some embodiments, the cancer is selected from the group consisting of: Adrenal Gland Tumor, Ampulla of Vater Tumor, Biliary Tract Tumor, Bladder/Urinary Tract Tumor, Bone Tumor, Bowel Tumor, Breast Tumor, CNS/Brain Tumor, Cervix Tumor, Esophagus/Stomach Tumor, Eye Tumor, Head and Neck Tumor, Kidney Tumor, Liver Tumor, Lung Tumor, Lymphoid Tumor, Myeloid Tumor, Other Tumor, Ovary/Fallopian Tube Tumor, Pancreas Tumor, Penis Tumor, Peripheral Nervous System Tumor, Peritoneum Tumor, Pleura Tumor, Prostate Tumor, Skin Tumor, Soft Tissue Tumor, Testis Tumor, Thymus Tumor, Thyroid Tumor, Uterus Tumor, and Vulva/Vagina Tumor. In some embodiments, the cancer is selected from the group consisting of: Adrenal Gland Tumor, Ampulla of Vater Tumor, Biliary Tract Tumor, Bladder/Urinary Tract Tumor, Bone Tumor, Bowel Tumor, Breast Tumor, CNS/Brain Tumor, Cervix Tumor, Esophagus/Stomach Tumor, Eye Tumor, Head and Neck Tumor, Kidney Tumor, Liver Tumor, Lung Tumor, Lymphoid Tumor, Myeloid Tumor, Other Tumor, Ovary/Fallopian Tube Tumor, Pancreas Tumor, Penis Tumor, Peripheral Nervous System Tumor, Peritoneum Tumor, Pleura Tumor, Prostate Tumor, Skin Tumor, Soft Tissue Tumor, Testis Tumor, Thymus Tumor, Thyroid Tumor, Uterus Tumor, Vulva/Vagina Tumor, Adrenocortical Adenoma, Adrenocortical Carcinoma, Pheochromocytoma, Ampullary Carcinoma, Cholangiocarcinoma, Gallbladder Cancer, Intracholecystic Papillary Neoplasm, Intraductal Papillary Neoplasm of the Bile Duct, Bladder Adenocarcinoma, Bladder Squamous Cell Carcinoma, Bladder Urothelial Carcinoma, Inflammatory Myofibroblastic Bladder Tumor, Inverted Urothelial Papilloma, Mucosal Melanoma of the Urethra, Plasmacytoid/Signet Ring Cell Bladder Carcinoma, Sarcomatoid Carcinoma of the Urinary Bladder, Small Cell Bladder Cancer, Upper Tract Urothelial Carcinoma, Urachal Carcinoma, Urethral Cancer, Urothelial Papilloma, Adamantinoma, Chondroblastoma, Chondrosarcoma, Chordoma, Ewing Sarcoma, Giant Cell Tumor of Bone, Osteosarcoma, Anal Gland Adenocarcinoma, Anal Squamous Cell Carcinoma, Anorectal Mucosal Melanoma, Appendiceal Adenocarcinoma, Colorectal Adenocarcinoma, Gastrointestinal Neuroendocrine Tumors, Low-grade Appendiceal Mucinous Neoplasm, Medullary Carcinoma of the Colon, Small Bowel Cancer, Small Intestinal Carcinoma, Tubular Adenoma of the Colon, Adenomyoepithelioma of the Breast, Breast Ductal Carcinoma In Situ, Breast Fibroepithelial Neoplasms, Breast Lobular Carcinoma In Situ, Breast Neoplasm, NOS, Breast Sarcoma, Inflammatory Breast Cancer, Invasive Breast Carcinoma, Juvenile Secretory Carcinoma of the Breast, Metaplastic Breast Cancer, Choroid Plexus Tumor, Diffuse Glioma, Embryonal Tumor, Encapsulated Glioma, Ependymomal Tumor, Germ Cell Tumor, Brain, Meningothelial Tumor, Miscellaneous Brain Tumor, Miscellaneous Neuroepithelial Tumor, Pineal Tumor, Primary CNS Melanocytic Tumors, Sellar Tumor, Cervical Adenocarcinoma, Cervical Adenocarcinoma In Situ, Cervical Adenoid Basal Carcinoma, Cervical Adenoid Cystic Carcinoma, Cervical Adenosquamous Carcinoma, Cervical Leiomyosarcoma, Cervical Neuroendocrine Tumor, Cervical Rhabdomyosarcoma, Cervical Squamous Cell Carcinoma, Glassy Cell Carcinoma of the Cervix, Mixed Cervical Carcinoma, Small Cell Carcinoma of the Cervix, Villoglandular Adenocarcinoma of the Cervix, Esophageal Poorly Differentiated Carcinoma, Esophageal Squamous Cell Carcinoma, Esophagogastric Adenocarcinoma, Gastrointestinal Neuroendocrine Tumors of the Esophagus/Stomach, Mucosal Melanoma of the Esophagus, Smooth Muscle Neoplasm, NOS, Lacrimal Gland Tumor, Ocular Melanoma, Retinoblastoma, Head and Neck Carcinoma, Other, Head and Neck Mucosal Melanoma, Head and Neck Squamous Cell Carcinoma, Nasopharyngeal Carcinoma, Parathyroid Cancer, Salivary Carcinoma, Sialoblastoma, Clear Cell Sarcoma of Kidney, Renal Cell Carcinoma, Renal Neuroendocrine Tumor, Rhabdoid Cancer, Wilms’ Tumor, Fibrolamellar Carcinoma, Hepatoblastoma, Hepatocellular Adenoma, Hepatocellular Carcinoma, Hepatocellular Carcinoma plus Intrahepatic Cholangiocarcinoma, Liver Angiosarcoma, Malignant Nonepithelial Tumor of the Liver, Malignant Rhabdoid Tumor of the Liver, Undifferentiated Embryonal Sarcoma of the Liver, Combined Small Cell Lung Carcinoma, Inflammatory Myofibroblastic Lung Tumor, Lung Adenocarcinoma In Situ, Lung Neuroendocrine Tumor, Non-Small Cell Lung Cancer, Pleuropulmonary Blastoma, Pulmonary Lymphangiomyomatosis, Sarcomatoid Carcinoma of the Lung, Lymphoid Atypical, Lymphoid Benign, Lymphoid Neoplasm, Myeloid Atypical, Myeloid Benign, Myeloid Neoplasm, Adenocarcinoma In Situ, Cancer of Unknown Primary, Extra Gonadal Germ Cell Tumor, Mixed Cancer Types, Ovarian Cancer, Other, Ovarian Epithelial Tumor, Ovarian Germ Cell Tumor, Sex Cord Stromal Tumor, Acinar Cell Carcinoma of the Pancreas, Adenosquamous Carcinoma of the Pancreas, Cystic Tumor of the Pancreas, Pancreatic Adenocarcinoma, Pancreatic Neuroendocrine Tumor, Pancreatoblastoma, Solid Pseudopapillary Neoplasm of the Pancreas, Undifferentiated Carcinoma of the Pancreas, Penile Squamous Cell Carcinoma, Ganglioneuroblastoma, Ganglioneuroma, Nerve Sheath Tumor, Neuroblastoma, Peritoneal Mesothelioma, Peritoneal Serous Carcinoma, Pleural Mesothelioma, Basal Cell Carcinoma of Prostate, Prostate Adenocarcinoma, Prostate Neuroendocrine Carcinoma, Prostate Small Cell Carcinoma, Prostate Squamous Cell Carcinoma, Aggressive Digital Papillary Adenocarcinoma, Atypical Fibroxanthoma, Atypical Nevus, Basal Cell Carcinoma, Cutaneous Squamous Cell Carcinoma, Dermatofibroma, Dermatofibrosarcoma Protuberans, Desmoplastic Trichoepithelioma, Endocrine Mucin Producing Sweat Gland Carcinoma, Extramammary Paget Disease, Melanoma, Merkel Cell Carcinoma, Microcystic Adnexal Carcinoma, Porocarcinoma/Spiroadenocarcinoma, Poroma/Acrospiroma, Proliferating Pilar Cystic Tumor, Sebaceous Carcinoma, Skin Adnexal Carcinoma, Spiroma/Spiradenoma, Sweat Gland Adenocarcinoma, Sweat Gland Carcinoma/Apocrine Eccrine Carcinoma, Aggressive Angiomyxoma, Alveolar Soft Part Sarcoma, Angiomatoid Fibrous Histiocytoma, Angiosarcoma, Atypical Lipomatous Tumor, Clear Cell Sarcoma, Dendritic Cell Sarcoma, Desmoid/Aggressive Fibromatosis, Desmoplastic Small-Round-Cell Tumor, Epithelioid Hemangioendothelioma, Epithelioid Sarcoma, Ewing Sarcoma of Soft Tissue, Fibrosarcoma, Gastrointestinal Stromal Tumor, Glomangiosarcoma, Hemangioma, Infantile Fibrosarcoma, Inflammatory Myofibroblastic Tumor, Intimal Sarcoma, Leiomyoma, Leiomyosarcoma, Liposarcoma, Low-Grade Fibromyxoid Sarcoma, Malignant Glomus Tumor, Myofibroma, Myofibromatosis, Myopericytoma, Myxofibrosarcoma, Myxoma, Paraganglioma, Perivascular Epithelioid Cell Tumor, Pseudomyogenic Hemangioendothelioma, Radiation-Associated Sarcoma, Rhabdomyosarcoma, Round Cell Sarcoma, NOS, Sarcoma, NOS, Soft Tissue Myoepithelial Carcinoma, Solitary Fibrous Tumor/Hemangiopericytoma, Synovial Sarcoma, Tenosynovial Giant Cell Tumor Diffuse Type, Undifferentiated Pleomorphic Sarcoma/Malignant Fibrous Histiocytoma/High-Grade Spindle Cell Sarcoma, Non-Seminomatous Germ Cell Tumor, Seminoma, Sex Cord Stromal Tumor, Testicular Lymphoma, Testicular Mesothelioma, Thymic Epithelial Tumor, Thymic Neuroendocrine Tumor, Anaplastic Thyroid Cancer, Hurthle Cell Thyroid Cancer, Hyalinizing Trabecular Adenoma of the Thyroid, Medullary Thyroid Cancer, Oncocytic Adenoma of the Thyroid, Poorly Differentiated Thyroid Cancer, Well-Differentiated Thyroid Cancer, Endometrial Carcinoma, Gestational Trophoblastic Disease, Other Uterine Tumor, Uterine Sarcoma/Mesenchymal, Germ Cell Tumor of the Vulva, Mucinous Adenocarcinoma of the Vulva/Vagina, Mucosal Melanoma of the Vulva/Vagina, Poorly Differentiated Vaginal Carcinoma, Squamous Cell Carcinoma of the Vulva/Vagina, and Vaginal Adenocarcinoma.

In some embodiments, (iii) comprises applying the prediction module to at least treatment features of the set of treatment options to determine the probabilistic predictions of the clinical outcomes of the set of treatment options. In some embodiments, the treatment features comprise attributes of a surgical intervention, a drug intervention, a targeted intervention, a hormonal therapy intervention, a radiotherapy intervention, or an immunotherapy intervention. In some embodiments, the treatment features comprise the attributes of the drug intervention, wherein the attributes of the drug intervention comprise a chemical structure or a biological target of the drug intervention.

In some embodiments, (iii) comprises applying the prediction module to at least interaction terms between the clinical data of the subject and the treatment features of the set of treatment options to determine the probabilistic predictions of the clinical outcomes of the set of treatment options.

In some embodiments, the clinical outcomes having future uncertainty comprise a change in tumor size, a change in patient functional status, a time-to-disease progression, a time-to-treatment failure, overall survival, or progression-free survival. In some embodiments, the clinical outcomes having future uncertainty comprise the change in tumor size, as indicated by cross section or volume. In some embodiments, the clinical outcomes having future uncertainty comprise the change in patient functional status, as indicated by ECOG, Karnofsky, or Lansky scores.

In some embodiments, the probabilistic predictions of clinical outcomes of the set of treatment options comprise statistical distributions of the clinical outcomes of the set of treatment options. In some embodiments, (iii) further comprises determining a statistical parameter of the statistical distributions of the clinical outcomes of the set of treatment options. In some embodiments, the statistical parameter is selected from the group consisting of a median, a mean, a mode, a variance, a standard deviation, a quantile, a measure of central tendency, a measure of variance, a range, a minimum, a maximum, an interquartile range, a frequency, a percentile, a shape parameter, a scale parameter, and a rate parameter. In some embodiments, the statistical distributions of the clinical outcomes of the set of treatment options comprise a parametric distribution selected from the group consisting of a Weibull distribution, a log logistic distribution, or a log normal distribution, a Gaussian distribution, a Gamma distribution, and a Poisson distribution.

In some embodiments, the probabilistic predictions of clinical outcomes of the set of treatment options are explainable based on performing a query of the probabilistic predictions.

In some embodiments, the method further comprises applying a training module that trains the trained machine learning model. In some embodiments, the trained machine learning model is trained using a plurality of disparate data sources. In some embodiments, the training module aggregates datasets from the plurality of disparate sources, wherein the datasets are persisted in a plurality of data stores, and trains the trained machine learning model using the aggregated datasets. In some embodiments, the plurality of disparate sources is selected from the group consisting of clinical trials, case series, individual patient cases and outcomes data, and expert opinions.

In some embodiments, the training module updates the trained machine learning model using the probabilistic predictions of the clinical outcomes of the set of treatment options generated in (iii). In some embodiments, updating is performed using a Bayesian update or a maximum likelihood algorithm.

In some embodiments, the trained machine learning model is selected from the group consisting of a Bayesian model, a support vector machine (SVM), a linear regression, a logistic regression, a random forest, and a neural network. In some embodiments, the trained machine learning model comprises a multilevel statistical model that accounts for variation at a plurality of distinct levels of analysis. In some embodiments, the multilevel statistical model accounts for correlation of subject-level effects across the plurality of distinct levels of analysis.

In some embodiments, the multilevel statistical model comprises a generalized linear model. In some embodiments, the generalized linear model comprises use of the expression:

$$η = X ⋅ β + Z ⋅ u$$

, wherein η is a linear response, X is a vector of predictors for treatment effects fixed across subjects, β is a vector of fixed effects, Z is a vector of predictors for subject-level treatment effects, and u is a vector of subject-level effects. In some embodiments, the generalized linear model comprises use of the expression: y = g⁻¹(η), wherein η is a linear response, g is an appropriately chosen link function from observed data to the linear response, and y is an outcome variable of interest.

In some embodiments, (iii) comprises applying a plurality of iterations of the prediction module to determine the probabilistic predictions of the clinical outcomes of the set of treatment options.

In some embodiments, the method further comprises using a parsing module to identify relevant features of the clinical data of the subject, the set of treatment options, and/or interaction terms between the clinical data of the subject and the treatment features of the set of treatment options. In some embodiments, the parsing module identifies relevant features by matching against a feature library.

In some embodiments, the method further comprises generating an electronic report comprising the probabilistic predictions of clinical outcomes of the set of treatment options. In some embodiments, the electronic report is used to select a treatment option from among the set of treatment options based at least in part on the probabilistic predictions of clinical outcomes of the set of treatment options. In some embodiments, the selected treatment option is administered to the subject. In some embodiments, the method further comprises applying the prediction module to outcome data of the subject that is obtained subsequent to administering the selected treatment option to the subject, to determine updated probabilistic predictions of the clinical outcomes of the set of treatment options.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 depicts the high-level architecture of a system of the present disclosure.

FIG. 2 shows four charts of time-series predictions of tumor load for brain cancer subjects, overlaid with actual tumor progression.

FIG. 3 shows one of the charts of FIG. 2 in greater detail.

FIG. 4 depicts one embodiment of a prediction module for tumor load and progression-free survival.

FIG. 5 illustrates the learning loop with subject outcomes data.

FIG. 6 depicts an interface showing summary subject data, including treatments, biomarkers, and outcomes data.

FIG. 7 illustrates the learning loop using expert survey data.

FIG. 8 may be a screenshot of an interactive tool for experts to provide feedback on subject cases.

FIG. 9 may be another screenshot of an interactive tool for experts to provide feedback on subject cases.

FIG. 10 illustrates the learning loop using clinical trial data as the source of new knowledge.

FIG. 11 shows a graph of brain cancer subjects’ response (progression-free survival) to irinotecan vs. all treatments.

FIG. 12 shows a computer system 1201 that may be programmed to implement methods of the disclosure.

[0074.1] FIG. 13 shows an example workflow of a method 1300.

### DETAILED DESCRIPTION

While various embodiments of the invention have been shown and described herein, it will be obvious to those skilled in the art that such embodiments may be provided by way of example only. Numerous variations, changes, and substitutions may occur to those skilled in the art without departing from the invention. It may be understood that various alternatives to the embodiments of the invention described herein may be employed.

As used in the specification and claims, the singular form “a”, “an”, and “the” include plural references unless the context clearly dictates otherwise.

As used herein, the term “subject,” generally refers to an entity or a medium that has testable or detectable genetic information. A subject can be a person, individual, or patient. A subject can be a vertebrate, such as, for example, a mammal. Non-limiting examples of mammals include humans, simians, farm animals, sport animals, rodents, and pets. A subject can be a person that has a cancer or may be suspected of having a cancer. The subject may be displaying a symptom(s) indicative of a health or physiological state or condition of the subject, such as a cancer of the subject. As an alternative, the subject can be asymptomatic with respect to such health or physiological state or condition.

Physicians engaged in precision oncology may integrate an overwhelming amount of information from publications and from their own experience. For example, as of the end of 2019, PubMed reports 19,748 publications matching the term “breast cancer” in the past year alone, and the same search for open, recruiting studies in ClinicalTrials.gov returns 1,937 studies. Therefore, practitioners, such as oncologists, may face challenges in reading all these materials, determining which may be most relevant, and synthesizing the whole of the data into relevant predictions for patient outcomes.

Oncologists fighting less common cancers may be potentially a worse situation; instead of being overwhelmed, they may have only a few relevant publications, and may have seen only a small number of similar cases. Here, successful prediction of patient outputs may depend on prior information gleaned from experts in similar, but not exactly the same, disease states.

Importantly, prediction may not be an exact science. Every patient may respond differently, due to a multitude of unknowns; it may be difficult or even impossible to fully model patients and their disease states, or the complete set of interactions between patients and their treatment regimens.

For some cancers, such as chronic myelogenous leukemia, the level of uncertainty may be relatively low; patients may almost universally receive tyrosine kinase receptor inhibitors, and the response characteristics may be relatively well-known. But for most cancers, and for many late-stage cancers, the number of unknown variables may far outnumber the number of known characteristics. In these cases, the sum of effects from the unknown variables may exceed the effects from known treatments. This may require probabilistic reasoning in order to devise an effective rational treatment strategy.

Thus, there remains a need for automated intelligent systems and methods that acquire and structure knowledge from a diverse array of sources, such as clinical trials, case series, individual patients cases and outcomes data, and expert opinions, such that such information may be used to predict, for a given patient, what the probable range of outcomes might be, over time, for a given treatment. Furthermore, such predictions may be explainable to a physician or scientist who queries the system for such a prediction; in contrast, a “black box” that provides answers without rationales may not instill confidence.

In light of the needs above, the present disclosure provides systems and methods for precision oncology using multilevel Bayesian models, which may effectively address challenges faced by physicians when treating patients with complex disease etiologies, such as cancer. Systems and methods of the present disclosure may be used to predict various measures of patient outcomes for particular patients under different treatment regimens. The systems and methods may be capable of learning from a diverse range of information sources, including individual patient outcomes observed outside of randomized trials (in other words, “real world evidence” or RWE) as well as other sources, such as expert surveys and summary statistics from clinical trials. The learning process may occur via a training module, which presents this data in a learning loop to a multilevel model module, which may be a combination of a Bayesian model and database.

Once the multilevel model module has been conditioned on such source data, it may be used in conjunction with a prediction module to predict outcomes for new patients under different treatment choices and provide a measure of the uncertainty of these predictions. These predictions may be probabilistic in nature, in that they represent a distribution of possible outcomes (e.g., in contrast to a single outcome).

A key advance may be that the multilevel model’s structure bears an understandable relationship to the domain, and to the types of inputs and outputs oncologists may expect. This structure may help users of systems and methods of the present disclosure to understand how the predictions and uncertainty therein may be derived, rather than treating the results as “black box” predictions. This level of explainability may be critical, for example, for certification of medical devices that rely on Artificial Intelligence and Machine Learning.

The model may be constructed or improved by a training process and a prediction process. For both of these tasks, the user may need to provide a list of relevant patient features (e.g., biomarkers), a list of relevant treatment features, and a list of possible interactions between features. Patient features (biomarkers) may include, but may be not limited to: somatic mutations (e.g., which may provide information about the cancer tumor itself); information about mutational burden (e.g., total number of mutations or number of mutations per million base pairs); germline genetic mutations (e.g., which may indicate higher risk of getting cancer, such as the BRCA1 and BRCA2 mutations); specific protein levels (e.g., if the protein ERCC1 may be present, then platinum-based chemotherapies may be not likely to be effective; other proteins of interest include certain enzymes, antibodies, and cytokines).

Treatment features may describe the various attributes of the treatments, such as whether it involves surgery, radiation, or may be a biochemical intervention. Each of these may be further subdivided. For example, surgical interventions may be divided into partial and total resections, exploratory biopsies, etc. Radiation may be described by wavelength, duration, burstiness, etc. For biochemical interventions, there may be multiple hierarchies, forming a lattice-like representation, of attributes that may describe the chemical structure, biological targets, and other attributes of the compounds. For example, the following hierarchy may be used, as described by Espinosa et al., “Classification of anticancer drugs—a new system based on therapeutic targets” CANCER TREATMENT REVIEWS 2003; 29: pp. 515-523, which may be incorporated by reference herein in its entirety:
Chemotherapy
   o Alkylators
   o Antibiotics
   o Antimetabolites
   o Topoisomerases inhibitors
   o Mitosis inhibitors
   o Other
Hormonal therapy
   o Steroids
   o Anti-estrogens
   o Anti-androgens
   o LH-RH analogs
   o Anti-aromatase agents
Immunotherapy
   o Interferon
   o Interleukin 2
   o Vaccines

This feature classification may be further refined, for example, down to the level of specific genes or pathways targeted by specific drugs (e.g., MEK, ERK, or p53).

The concatenation of a list of biomarkers, treatment features, and interactions terms between these features may specify a set of predictors for the model. In addition to identifying the predictors, a user of the system of the present disclosure may specify the desired treatment outcomes of interest, to be predicted by the system. These outcomes may include, but may be not limited to: a change in tumor size (e.g., as measured in cross section, or in volumetric estimation); a change in patient functional status (e.g., ECOG, Karnofsky, or Lansky scores); a time-to-disease progression; a time-to-treatment failure; an overall survival; and a progression-free survival.

With the set of predictors and a desired set of outcomes, the system may then generate a “predictive model,” which may be a forward simulation from a set of given predictors to the set of desired outcomes. Because these simulations may be stochastic in nature, they may involve a plurality of iterations and produce a statistical distribution of possible treatment outcomes. The outcome predictions may be communicated as summary statistics of this distribution, such as the mean and standard deviation for continuous outcomes, shape/scale parameters of the distribution, or the frequency of specific cases for discrete outcomes (e.g. a rate parameter).

The predictive model may be a generalized linear multilevel model. As such, the expected outcome of the generalized linear model may be a linear combination of predictor variables, under an appropriate transformation of the outcome variable. Multilevel models may be statistical models that account for variation at multiple levels of analysis. For instance, the model may measure the size of a subject’s tumors each month for several months after treatment. Variation in the size of a subject’s tumor at a particular time may be due to either the characteristics specific to the subject (e.g., having a more or less aggressive tumor) or from the time relative to the start of treatment. Additionally, such a model may consider subject-level effects on the time-to-disease progression or death to be additional effects on the survival of subjects which, while they may be correlated with predictors, may not be fixed across subjects when conditioned on the predictors. Models that fail to account for correlation in data from different levels of analysis may underestimate the uncertainty of model predictions.

To perform the learning task, a learning module may update the state of the predictive model by conditioning it on new data. This new data may take the form of any treatment outcomes data that may be predicted by the predictive model or by summary statistics derived from the predictive model. The state representation of the model may be any representation of a probability distribution over such model parameters, such as a finite number of samples from the distribution, summary statistics of the distribution, or hyperparameters describing a particular instance of a parametric family of probability distribution functions. While the learning task may be considered a form of a Bayesian update, such an updating procedure may use techniques from frequentist statistics, such as maximum likelihood algorithms to derive new model parameters.

Improved systems and methods for predicting treatment outcomes may comprise improvements in the application of subject-specific biological features and/or the application of black box machine learning algorithms such as neural networks to the task of generating predictions of outcomes.

For example, systems and methods for predicting treatment outcomes may be improved in the application of subject-specific biological features. For example, genetic sequencing of a subject’s tumor may reveal mutations in known oncogenes (e.g., genes that have the potential to cause cancer). The presence or absence of mutations in these genes may be shown in randomized controlled trials to affect the efficacy of particular drugs that target proteins in related metabolic pathways. Methods for applying this knowledge may comprise use of decision trees whose decision criteria may be set by published studies. While these methods may provide clear guidance on applying the predictions, there may be little or no quantification of uncertainty in the predictions. Such uncertainty quantification may naturally arise in a Bayesian outcomes model, in which uncertainty may be expressed as the variance in the distribution of predicted outcomes. An additional challenge that such methods face may be that they require expensive clinical studies in order to discover new rules for achieving better outcomes, with results that may take years or even decades to be disseminated to widespread practice in the community. In contrast, the Bayesian outcomes model presented herein may be updated with multiple sources, including individual subject data, existing clinical trial data, and expert surveys, and it may be done in a timely fashion.

As another example, systems and methods for predicting treatment outcomes may be improved in the application of black box machine learning algorithms such as neural networks to the task of generating predictions of outcomes. Such algorithms may achieve high predictive accuracy but may require large datasets to make sensible predictions. Thus, they may not generalize well beyond the scope of data that the model has been trained on. Since many cancers may be rare, and many subjects present unique circumstances, training such networks may be difficult.

Training such systems may face challenges from the “large p, small n” problem. That is, there may be a very large number of parameters that may be fitted compared to the number of data points available for training. As an example, consider the size of the human genome and the number of possible mutations it may harbor, in relation to the number of childhood brain cancer subjects. The problems and challenges associated with potential overfitting may be enormous.

In addition, these algorithms may be difficult for domain experts to interpret and critique. Aside from hindering the adoption of such algorithms by care providers, the lack of interpretability may make it difficult to debug these algorithms. The same problems with non-explainability of the algorithms may make it difficult when it comes to consideration of systems utilizing such algorithms for certification as software medical devices.

Thus, there remains a need for systems and methods that may predict measures of subject outcomes using a relative paucity of data, which may handle uncertainty in prediction, and which may explain the outcomes in terms of features that a physician may use to describe the subject’s condition, such that a physician may understand why the system came to the conclusion that was determined.

In a generalized linear multilevel model, the linear response to a treatment may be described by the expression:

$$η = X ⋅ β + Z ⋅ u$$

where η may be the linear response, X is a vector of predictors for treatment effects fixed across subj ects, β is a vector of fixed effects, Z is a vector of predictors for subject-level treatment effects, and u is a vector of subject-level effects. Z may comprise any subset of predictors from X, indexed by subject. The subject-level effects parameters may be asserted or assumed to be drawn from a zero-centered multivariate normal distribution. These subject-level effects may have the interpretation as the variation in outcomes in subjects beyond those due to measured predictor variables.

The expectation of the linear response may be described by the expression:

$$y = {g}^{- 1} \left(η\right)$$

where g is an appropriately chosen link function from the observed data to the linear response and y is the outcome variable of interest. The distribution about the expected value may be chosen to match the range of the outcome space, such as a normal distribution for continuous outcomes or a categorical distribution for discrete outcomes. Other outcomes, such as time-to-event outcomes, may use a more specialized distribution such as a Weibull, log logistic, or log normal distribution. Such distributions with additional shape or scale parameters beyond η may introduce additional linear dependence on predictor variables and subject-level variables.

Importantly, the prediction model provided herein may not be stateless. It may accumulate knowledge over time, by being trained via a set of training inputs, and/or by learning from every example it may be presented with. Further, since the effects parameters may not be scalar parameters, but rather may be drawn from distributions, it may be possible to provide prior estimates of degrees of confidence or degrees of belief in certain effects, even if there have been no concrete cases yet available to examine (e.g., in a case where there have been in vitro experiments but there has not yet been in vivo usage of a drug, there may only be expert opinion to draw from at the moment).

The machinery that surrounds the prediction model may be organized into several modules that perform different functions, depending on whether the system may be being trained with training data or being asked to predict the outcomes for a specific subject.

At a simple level of abstraction, the systems and methods of the present disclosure may be used in different modes. When used in “training mode,” the system may be presented with multiple training examples, each of which comprises a subject case description and the actual treatment outcome. This data may be used to train an internal model (e.g., through one or more iterations), but may produce no output (other than for debugging purposes and monitoring purposes).

When used in “prediction mode,” the system may be presented with a single subject case at a time. The system may then use the model to produce predicted outcomes, which describe the expected trajectory of a test subject on the proposed treatment regimen. These outcomes may be time-dependent and probabilistic in nature.

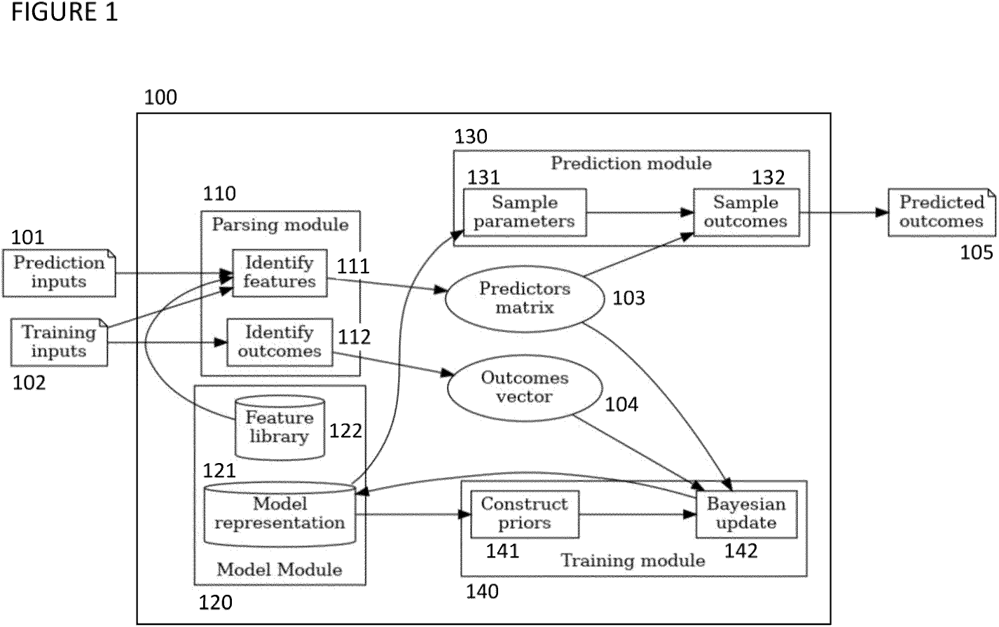

FIG. 1 displays the relationships among four modules of a system or method of the present disclosure. It shows the architecture of how these modules interact, as well as the major component within each module.

The system 100 comprises four modules: the parsing module 110, the model module 120, the prediction module 130, and the training module 140. In “training mode,” the system may be presented with training inputs 102, which may be training examples which have both input and outcome information. These training inputs may be used to update the internal model representation 121, and may be the way by which the system learns.

Another way the system may be used may be in “prediction mode.” In this mode, the system may be provided only features of a particular subject and treatment regimen in the prediction inputs 101. The system may then use the knowledge stored in the model representation 121, along with other parts of the system, and may generate predicted outcomes 105 therefrom. These predictions may not necessarily be exact values, but may be expected values with credible intervals associated with them.

For performing the prediction task, the user of the system may provide prediction inputs (e.g., subject case descriptions) to the parsing module. The parsing module may identify relevant biomarkers, treatments, and interaction terms by matching against the feature library 122. This identification process may produce a matrix of predictors 103, whose rows represent different treatment options and whose columns represent different feature variables that may be associated with variation in outcomes (alternatively, without loss of generality, rows may represent different feature variables that may be associated with variation in outcomes, and columns may represent different treatment options). The prediction module may iteratively draw sample parameters 131 from the model representation, then may use these sampled parameters with the predictor matrix to draw a sample of outcomes 132 under each treatment option. This iterative process may be repeated to build a larger sample of predicted outcomes 105 under each treatment option.

For performing the training task, the user of the system may provide training inputs 102 (e.g., subject treatment outcomes data, expert survey data, or clinical trial data) to the parsing module 110. The parsing module 110 may identify relevant biomarkers, treatments, and interaction terms by matching against the feature library 122. This identification process may produce a matrix of predictors 103, whose rows represent different treatment options and whose columns represent different feature variables that may be associated with variation in outcomes (alternatively, without loss of generality, rows may represent different feature variables that may be associated with variation in outcomes, and columns may represent different treatment options). In addition, the parsing module 110 may identify treatment outcomes from the training inputs, and the parsing module 110 may produce a vector of outcomes 104. The training module may read the current model representation to construct a Bayesian prior distribution 141. The training module may then take these priors, and may use the predictors matrix and outcomes vector to perform a Bayesian update 142. This updating process may produce an updated model representation, which may be stored in place of the previous model representation 121.

While some embodiments of the present disclosure utilize Bayesian modeling to perform an update of internal model state, the same task may be performed using frequentist statistical techniques. For example, the Bayesian formulation may be simpler to use; however, limitation of the discussion may in no way be interpreted as a limitation of the present disclosure.

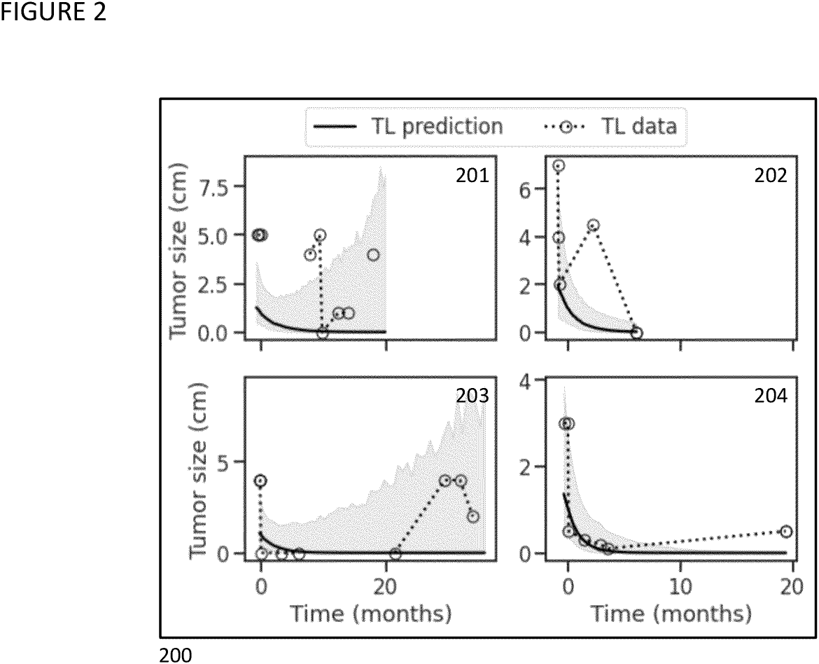

FIG. 2 illustrates a set of four predictions 200 for the outcome of tumor load over time, after the model has already been trained on a data set of training data. Each of the four panels 201, 202, 203, and 204 that make up the set, displays a prediction for a different subject for whom the model generated the tumor load (TL) prediction, as predicted size vs. number of months from present time into the future. Notably, these graphs show both the predictions and the actual observed tumor loads; in actual use, a physician may see only the predictions, because the future tumor loads may not yet have been measured.

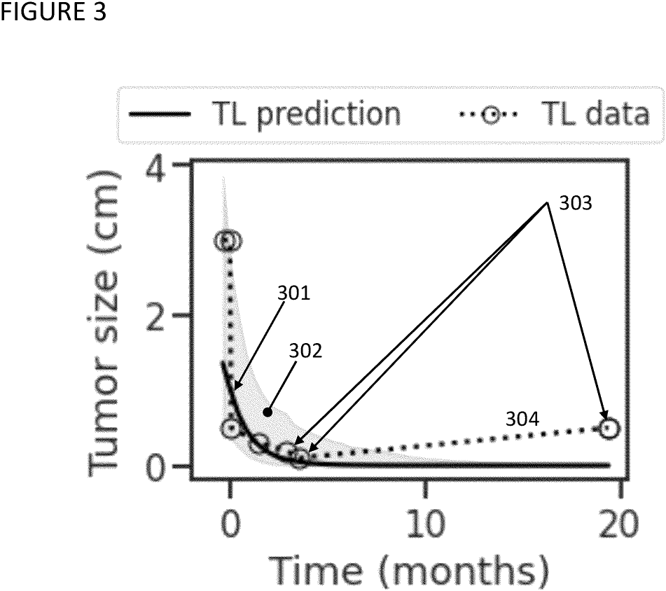

The outcome prediction shown in panel 204 may be enlarged and shown in FIG. 3 so the details may be explained. The system may predict a distribution of possible tumor loads (TLs). The center of the distribution may be illustrated by the dark line 301, while the gray area 302 may denote the region between the 16th and 84th credible intervals. At a later date (in this case, 20 months after the prediction may be made), the actual data may be overlaid onto the predicted data. Actual measurements of tumor load may be shown by the circles, three of which may be pointed to by lines 303. All the circles may be connected by dotted line 304.

Returning to FIG. 1, the components and sub-tasks that make up a system of the present disclosure may be described in more detail, so that the generation of these prediction graphs may be fully understood.

### Model Module

The model module 120 may comprise model representation 121 and feature library 122. The model representation may be a database which comprises a record of model parameter distributions for each outcome type (e.g., time-to-disease progression, change in tumor load, change in performance status). These parameter distributions may be stored either as a finite number of samples from the distribution of interest or as hyperparameters of some parametric probability distribution (note that “hyperparameter” here may be used in the Bayesian sense, to refer to parameters that describe a particular probability distribution, as compared to the machine learning sense of parameters that may be tweaked to tune how an algorithm runs). The feature library may be another database comprising a list of treatment options, a list of biomarkers, and a list of interaction terms that reference entries in the treatment and biomarker lists. All of this information may be used in creating the predictors matrix 103, which may be used in intermediate calculations.

### Parsing Module

The parsing module 110 may perform the following sub-tasks: upon being presented with training input data 102, the “identify features” subsystem 111 constructs the predictors matrix 103, and the “identify outcomes” subsystem 112 constructs the outcomes vector 104. Additionally, “identify features” subsystem 111 constructs the predictors matrix 103 when presented with prediction inputs 101. Training input data may comprise multiple subject case descriptions associated with treatment outcomes. Prediction input data may comprise a single subject case description.

To construct a predictors matrix from training input data, the parsing module 110 may partition the training data by individual subjects, then may construct, for each subject, a vector of features by matching the individual subject’s case description to the list of features provided by the feature library in the model module. These feature row vectors may be concatenated to form a matrix of predictors (predictors matrix 103). To construct the outcomes vector 104, the parsing module may similarly partition the training data by individual subjects, then may associate each subject with a treatment outcome.

To construct a predictors matrix 103 from prediction inputs, the parsing module 110 may create a copy of the subject case description for each treatment option read from the feature library. Each treatment option may be associated with a copy of the case description. The parsing module 110 may take this set of case descriptions with hypothetical treatments, then for each hypothetical treatment, it may form a feature vector by matching against the biomarker, treatment, and interaction terms stored in the feature library 122. These feature row vectors may be concatenated to form a matrix of predictors, where the rows in this matrix represent different hypothetical treatment scenarios.

### Prediction Module

The prediction module 130 may generate predicted outcomes 105 under different treatment options. Treatment options may be represented as rows of the inputted predictors matrix. Because predictions may be probabilistic in nature, representing a distribution of possible outcomes, they may be generated by sampling distributions. Thus, the prediction module may first sample parameters 131 from the parameter distribution stored in the model representation, then the prediction module 130 may sample from the outcomes distribution 132, conditional on the previously sampled parameters. These two subsystems may repeat their processes one or more times, as necessary, to generate a representative distribution.

The process by which the particular features may be chosen may be manual. Alternatively, automatic generators based on, for example, natural language parsing of domain models or simple causal diagrams, may be used.

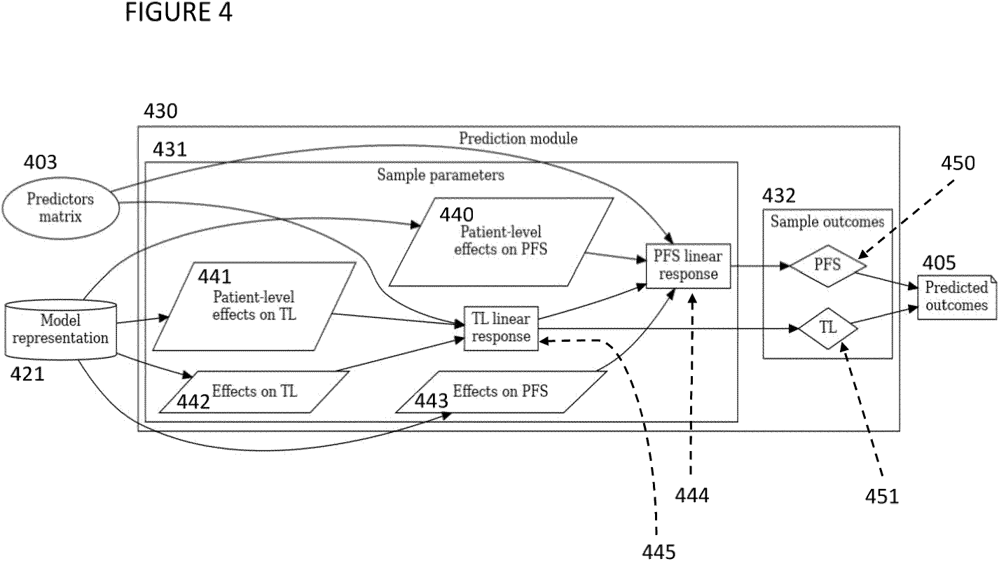

FIG. 4 depicts one such example, using a number of subject features and biomarkers, plus different treatments, to predict changes in tumor load (TL; e.g., the spatial extent of a subject’s solid tumor) and progression-free survival (PFS; e.g., the time to disease progression or death). In FIG. 4, predictors matrix 403 corresponds to predictors matrix 103 of FIG. 1, model representation 421 corresponds to model representation 121 of FIG. 1, prediction module 430 corresponds to prediction module 130 of FIG. 1, sample parameters 431 corresponds to sample parameters 131 of FIG. 1, sample outcomes 432 corresponds to sample outcomes 132 of FIG. 1, and predicted outcomes 405 corresponds to predicted outcomes 105 of FIG. 1.

The remaining components of FIG. 4 show further details that illustrate workings of this specific prediction module. A key assumption may be that TL may be highly likely to affect PFS, but PFS may be unlikely to affect TL.

The sample parameters module 431 may read the model representation 421 to fetch values for the following model parameters: effects on TL 442, subject-level effects on TL 441, effects on PFS 443, and subject-level effects on PFS 440. The predictors matrix 403 may be multiplied by the vector of effects on TL 442 and added to the product of the predictors matrix with the subject-level effects on TL to form the TL linear response 445 variable. The TL linear response may be used as an additional predictor along with the other predictors from the predictors matrix for calculating the PFS linear response 444 from the vector of effects on PFS 443 and subject-level effects on PFS 440.

The sample outcomes module 432 may take the linear responses for TL 451 and PFS 450, and draw a sample from the appropriate outcomes distribution. For this example, sample TL outcomes may be drawn from a LogNormal distribution whose location parameter may be specified by the TL linear response 445, and sample PFS outcomes may be drawn from a LogLogistic distribution whose location parameter may be specified by the PFS linear response 444. The sampled outcomes may be appended to the list of predicted outcomes.

Each subtask of parameter sampling and outcomes sampling may be independently repeated over some pre-specified number of iterations (e.g., 1,000 or 10,000) to generate a distribution of predicted outcomes. This predicted outcomes distribution may be summarized by e.g., mean and standard deviation statistics, which provide an indication of the expected outcome and the uncertainty, respectively.

The use of tumor load and progression-free survival as metrics of subject outcomes was provided for illustrative purposes only, and may be not intended to be limiting in any respect. Other metrics may also be created using similar approaches, such as, but not limited to: tumor markers (e.g., CA19-9); overall survival; performance scores (e.g., ECOG or Karnofsky Score), serious adverse events, and so forth.

### Training Module

Returning to FIG. 1, the training module 140 may be responsible for taking training inputs 102 from users of the system and turning them into stored knowledge in the model module 120. Training inputs may be first parsed using the parsing module to create a predictors matrix 103 and outcomes vector 104. The training module may then take as inputs the current model representation 120, predictors matrix, and outcomes vector. The training module may output an updated version of the model representation, which then replaces the model representation 121 in the model module. This new representation may be used for future prediction tasks. This loop of using the model representation with new inputs to create and update the model may be perform as the “learning loop.”

At the next level, the training module comprises a subsystem for constructing priors 141, and a subsystem for performing a Bayesian update 142. The subtask of construction priors may be done by either directly taking samples of model parameters from the model representation 121, or by reading the hyperparameters and functional form of the parameter distribution from the model representation. The Bayesian update process may be performed with a wide variety of algorithmic methods, such as Markov Chain Monte Carlo, Variational Bayesian Inference, and Approximate Bayesian Computation.

An example of such a Bayesian update algorithm may be a Markov Chain Monte Carlo procedure with Metropolis-Hastings proposals (however, other algorithms may be possible; this example may be not meant to be limiting):
1. Start from an initial set of model parameters drawn from the prior probability distribution, an empty chain of model parameters, a proposal distribution, and a desired number of samples.
2. Propose a new set of model parameters by using the proposal distribution conditioned on the current set of model parameters.
3. Evaluate the posterior probability density value (up to a normalizing constant) of both the current set of model parameters and the proposed set of model parameters, then calculate the acceptance ratio as the ratio of the proposed density to the current density.
4. Generate a random number between 0 and 1. If the random number may be less than the acceptance ratio, add the proposed parameters to the chain. Otherwise add the current parameters to the chain.
5. Repeat operations 2 to 4 until the length of the chain matches the desired number of samples.

In some embodiments, the system may “warm-up” the chain over some large number of iterations until the Markov Chain may be stationary, then may draw samples from the distribution until the desired number of samples may be reached. Metrics such as the autocorrelation time and the Gelman-Rubin convergence statistics may be used to assess the convergence of the algorithm.

The learning loop may be adapted or customized to deal with different types of informative prior information. For example, the system may learn from examples of subjects interacting with their care providers; this may be a case where treatment decisions may be made, and importantly, follow-up data on the subject’s outcome may be available. In another example, the system may learn from surveys of expert opinion; in this case, no subject outcome data may be available, but because the data comes from experts, the strength of the prior beliefs may be high. In another example, the system may learn from clinical trials data; in this case, data involve real subjects with rigorous controls. These three examples may be illustrative and may not be exhaustive. Numerous other examples of learning opportunities may be applied to systems and methods of the present disclosure.

All of these examples involve use of the parsing module, predictions matrix, outcomes vector, the training module and the model module, but arranged in slightly different ways, as may be illustrated herein.

### Interacting With Subjects and Their Care Providers

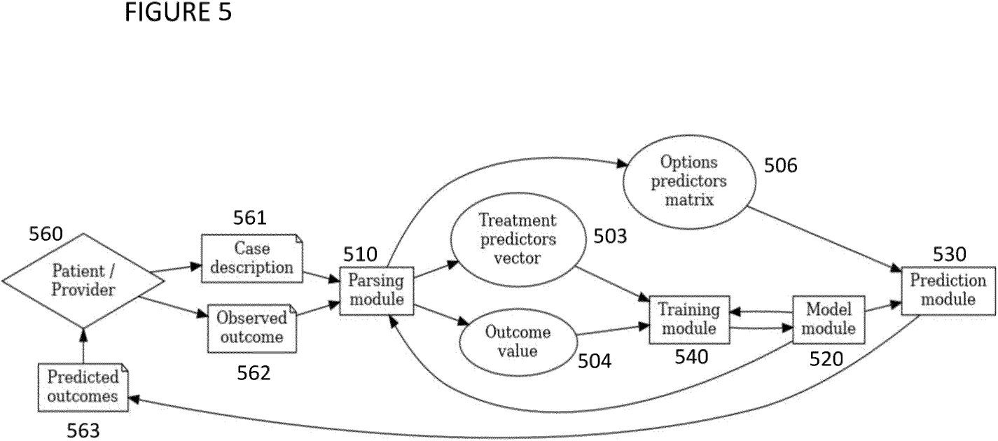

FIG. 5 displays an example of a process by which a subject and/or their care provider interacts with the system. In order to affect learning, it may be important to note that there may be two trips through the learning loop, each involving different components, as may be illustrated herein.

Initially, a subject and the subject’s provider (together, 560) may wish to use the system to decide on the best course of treatment. They may input a case description 561 (which corresponds to prediction inputs 101 in FIG. 1). This may be a textual description of the subject’s case. This may be parsed by the parsing module 510 (corresponding to module 110 of FIG. 1) to construct the treatment options predictors matrix 506 for the subject. This predictors matrix may contains rows for each possible treatment option. The prediction module may use the predictors matrix, and the model representation may be read from the model module to generate predicted outcomes under each treatment option. Since the system may be used in “prediction mode,” no training may be done.

In some embodiments, the options predictors matrix 506 may be generated (corresponding to predictors matrix 103 of FIG. 1), and the predictions module 530 may return the predicted outcomes 563 (corresponding to predicted outcomes 105 of FIG. 1), which may be returned to the subject or provider. This may be an example of a use of the system in “prediction mode.”

At this point, and separately from the system, the subject and provider may discuss the options available to them, make a treatment decision, and begin treatment. This may result in an outcome at some future date (e.g., an increase or decrease in the subject’s tumor by some measurable amount), and they may again use a system of the present disclosure at that future date to enter information about how well the treatment performed. This may be where learning takes place.

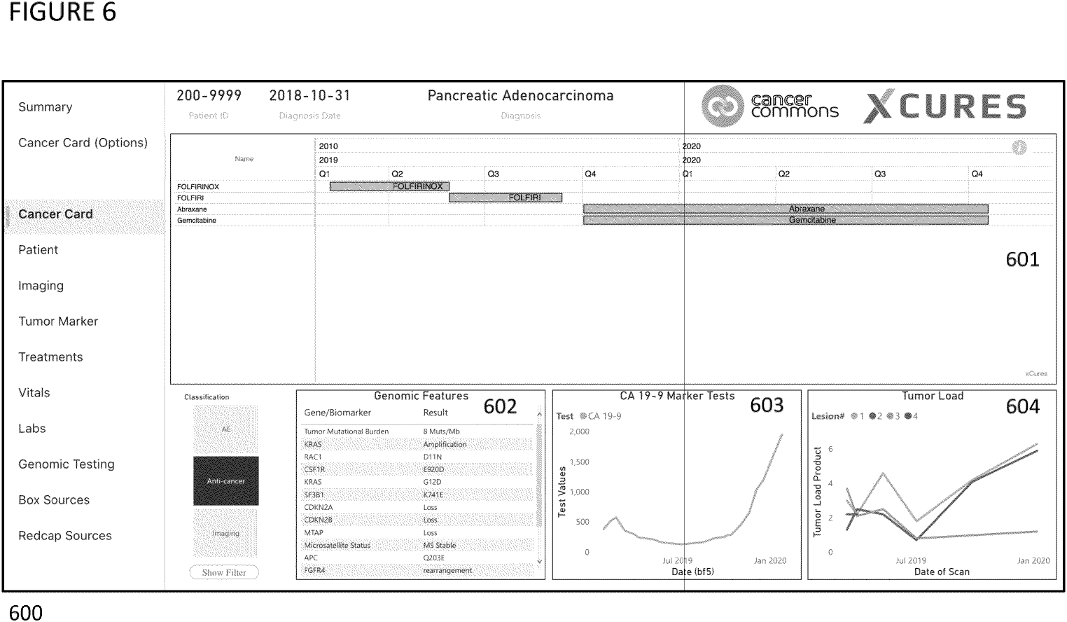

An example of data being entered and displayed in the system may be shown in FIG. 6, where the subject and doctor have stopped treatment of a heavily cytotoxic chemotherapy (FOLFIRINOX), due to the neuropathy caused by the platinum in the formulation. Screenshot 600 shows an interface where providers and subjects may see summary information, including cancer drug treatments 601, genomic information 602, certain biomarkers 603, and tumor load 604. In this example, the subject has decided the neuropathy may be too severe a side effect, and may be willing to accept the risk of increased tumor burden, even though it may be predicted by the system. As shown in panel 601, the subject’s treatment regimen may be switched to a mixture of gemcitabine and abraxane (less toxic, but less effective than FOLFIRINOX) in Q4 of 2019, and as may be seen in panels 603 and 604 respectively, the CA 19-9 biomarker rises, and two of three tumors begin to grow.

Returning to FIG. 5, in the learning phase, the subject and/or provider 560 may input the case description 561 and also the observed outcome 562 to the parsing module 510, which may construct a treatment predictors vector 503 (corresponding to predictors matrix 103 in FIG. 1) representing the chosen treatment, as well as a value representing the treatment outcome 504 (corresponding to outcomes vector 104 in FIG. 1). The reason vector 503 may be not a predictors matrix, as in the “prediction mode” usage before, may be that there may be only a single treatment, and hence the matrix collapses to a single row.

The training module 540 may take inputs 503 and 504, as well as the current model representation from the model module 520, to produce an updated model representation. This may complete the “learning loop”, in that the next subject that interacts with the system will receive better predictions from the system due to the updated model from the previous subject’s data.

### Learning From Expert Surveys

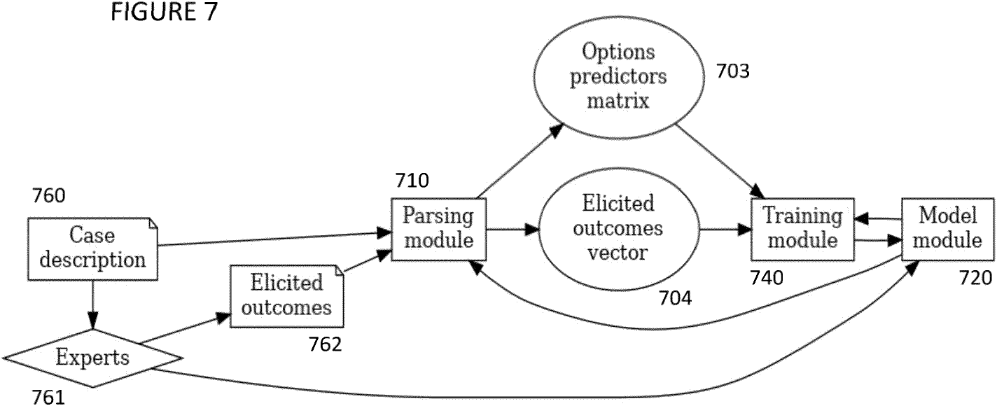

FIG. 7 illustrates how the system may interact with one or more biomedical experts to learn from their expertise. First, an expert or panel of experts 761 may be convened to discuss a subject’s case 760. The experts may be prompted to predict outcomes under different possible treatment options (elicited outcomes 762). The experts may be additionally prompted for any features of the subject’s case that were important for their decisions, and these features may be added to the feature library in the model module 720, if they are not already present.

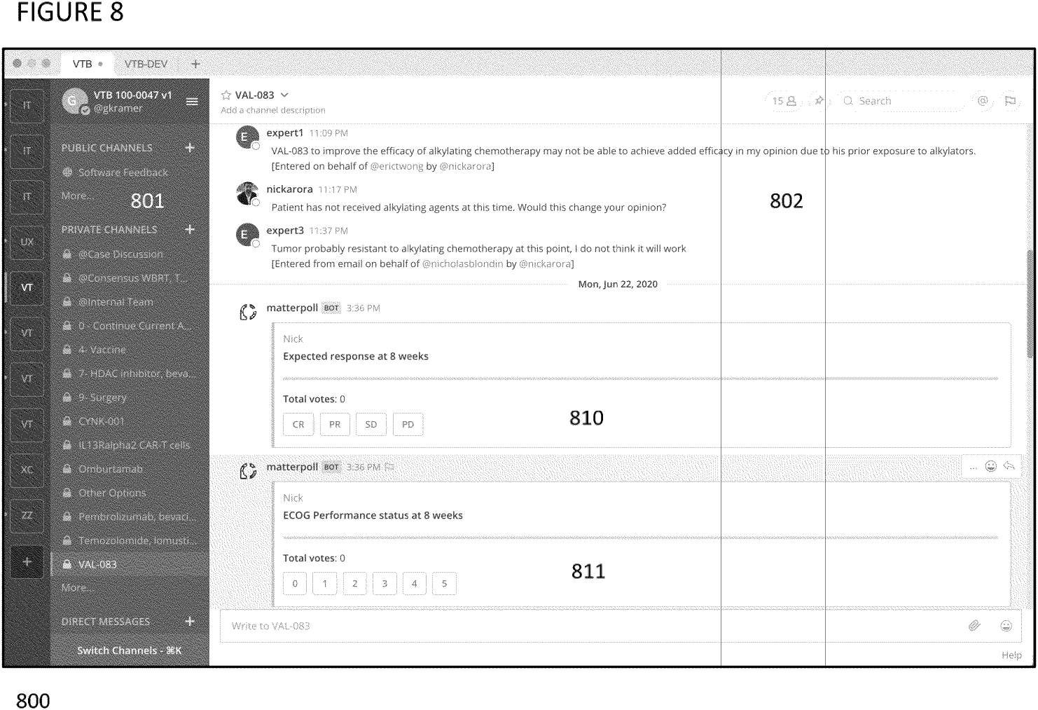

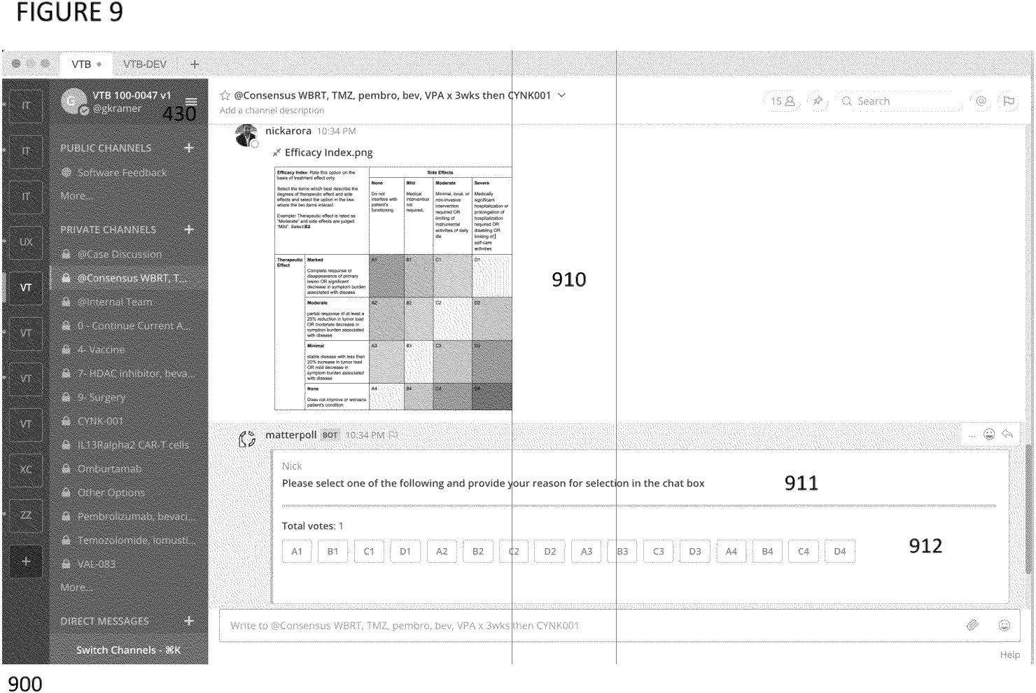

FIG. 8 and FIG. 9 show screenshots of a tool where experts may discuss clinical cases to discuss possible treatments, as well as input survey data in numerical format for use with a system of the present disclosure. In FIG. 8 (screenshot 800), side panel 801 shows how discussion may be organized along channels that allow discussion of the case itself, as well as each of the potential treatment options under consideration. The highlighted treatment option “VAL-083” may be the item under consideration, and may be the discussion to the right. Text 802 may be the tail end of a discussion among different experts regarding the efficacy and risks of this option. After the discussion, a series of polls 810 and 811 may be provided to allow experts to easily express their opinions in a standardized way. Question 810 asks what the response to the VAL-083 treatment may be expected to be at 8 weeks: complete response (CR); partial response (PR); stable disease (SD); or, progressive disease (PD). Question 811 asks for the subject’s expected ECOG performance score, on a scale from 0 to 5, at 8 weeks after treatment.

FIG. 9 illustrates another approach of obtaining numerical rankings from an expert. In the screenshot 900, there may be a matrix 910 with two axes, one with a range of therapeutic effects, and the other with a range of side effects. The best choices may have a great therapeutic effect with low side effects, and the worst choices may have a low therapeutic effect with significant adverse side effects. Experts may be prompted by a facilitator 911 to evaluate the particular therapy. Each box in the matrix may be labeled from A1 through D4, and experts may select their choices from multiple choice poll 912.

The results of these polls, along with natural language discussions that may be mined for rationales, may be stored in this tool, allowing results to be communicated to a system of the present disclosure, among other uses.

Returning to FIG. 7, the elicited outcomes from the experts, as well as the case description, may be processed by the parsing module 710 (corresponding to parsing module 110 of FIG. 1), which may match the case description against the feature library from the model module 720 (corresponding to model module 120 of FIG. 1) to generate an options predictors matrix 703 representing treatment options considered by the experts as well as a vector of elicited treatment outcomes 704 (these correspond to the predictors matrix 103 and outcomes vector 104 of FIG. 1). The training module 740 may take the options predictions matrix, the elicited outcomes vector, and the current model representation from the model module 720 to generate an updated model representation.

Note that this learning may be done purely based on the opinions of the experts, and not on any actual subject outcomes based on treatments. However, experts often have decades of experience, and may use lateral thinking and analogous reasoning to predict how previously unused combinations of therapies may work together, even in the absence of hard evidence.

### Learning From Clinical Trial Data

A simple reconfiguration of the system’s components may allow training of the model from data that has already been processed from groups of individual subjects, such as summary statistics from clinical trials data. More concretely, a clinical trial may describe the features of its subject sample, the treatments given to subjects, and the median progression-free survival in cohorts of subjects that received particular treatments.

To perform the training task in this scenario, some embodiments of the present disclosure apply an Approximate Bayesian Computation (ABC) method. In context, the ABC rejection sampling algorithm may be performed as follows.
1. Start with observed data that may be computable from individual subject outcomes data (e.g., summary statistics of individual outcomes), a specified prior probability distribution over model parameters, and a desired number of samples from the posterior probability distribution.
2. Sample parameters from the prior probability distribution.
3. Use the sampled parameters to generate data using the outcomes model.
4. Summarize the individual outcomes data following the same procedure as the observed data.
5. Compare the predicted outcomes summary statistics with the observed statistics; if the difference between the two values may be below some pre-specified threshold, then accept the sample from the prior probability distribution as a sample from the posterior distribution. Otherwise reject the prior sample.
6. Repeat operations 2 to 5 until the desired number of samples from the posterior probability distribution is reached.

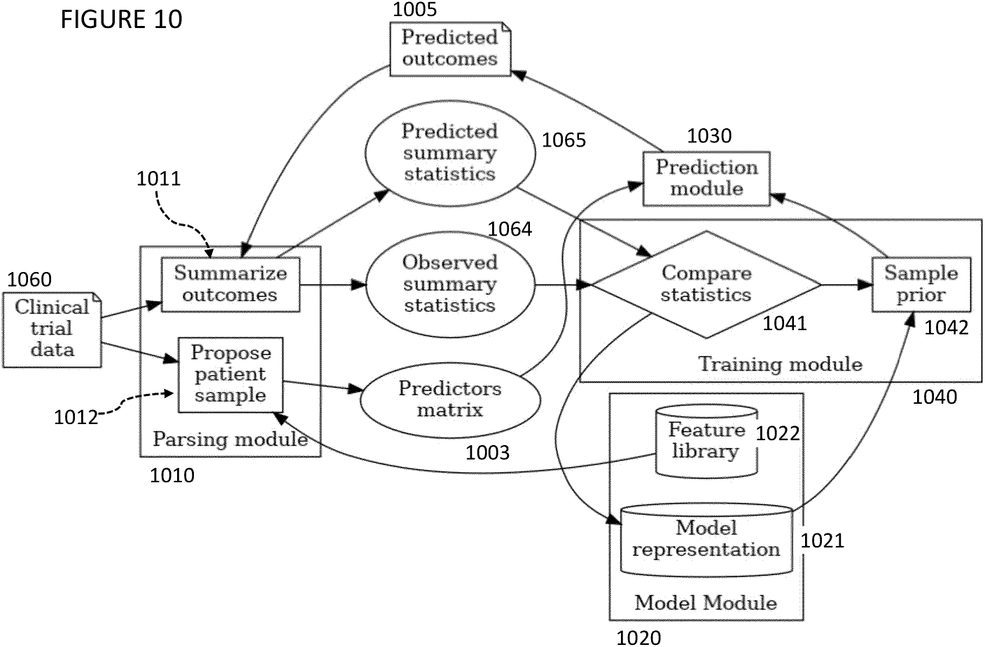

FIG. 10 depicts a configuration of the system in which the training module may be used to update the model from clinical trial data that lack information on individual subject outcomes. Data from a clinical trial 1060 may be input into the parsing module 1010 (this corresponds to training input 102 being input to parsing module 110 of the system in “training mode” in FIG. 1). The parsing module may perform two tasks with this data. First, the summarize outcomes subsystem 1011 may process input data from the trial to produce outcome summary statistics (observed summary statistics 1064). This operation may replace the identifying features operation 111 of FIG. 1.

The second operation may mark the beginning of the Approximate Bayesian Computation (ABC) loop. In this operation, which may be the propose subject sample operation 1012, the parsing module may match any inclusion or exclusion criteria and treatment arm descriptions from the clinical trial data against the feature library 1022 from the model module 1020 (corresponding to module 120 in FIG. 1) to propose a synthetic subject sample whose features may be consistent with the reported subject sample from the clinical trial. The output of this subsystem may be a predictors matrix 1003, whose rows correspond to different individual synthetic subjects that, as a whole, have biomarkers and treatments consistent with the clinical trial report.

Next, the training module 1040 (corresponding to module 140 in FIG. 1) may read the model representation 1021 in the model module 1020 to sample a set of parameters from the prior probability distribution. Prediction module 1030 (corresponding to module 130 in FIG. 1) may receive the prior parameter sample 1042 as well as the predictors matrix 1003 as inputs to produce a set of predicted outcomes 1005. The parsing module 1010 may receive these predicted outcomes and summarizes these outcomes to produce predicted outcome summary statistics 1065.

At this point, there may be observed summary statistics 1064 from the clinical trial, and predicted summary statistics 1065 from a synthetic subject population. Both the observed summary statistics and the predicted summary statistics may be fed to the compare statistics operation 1041 within the training module. On each ABC iteration, the training module may read the most recent model representation from the mode module. The comparator 1041 may compare the observed and predicted summary statistics, using a pre-specified threshold for how close these quantities need to be in order to be accepted.

If the observed and predicted summary statistics are close enough, then the training module may store the sampled set of prior parameters in the model representation, and the system may successfully exit the training loop. Otherwise, the training module may reject the current parameter sample and another ABC iteration may begin, which includes generating additional synthetic subject samples in the propose subject sample operation 1012, new predicted summary statistics 1065, and the training module comparing statistics again in operation 1041 to check for the ABC loop exit criteria.

### Detecting Subpopulations of Subjects

In multilevel modeling, the observed variation in outcomes may be assumed to be split between fixed effects from observed covariates and random effects that vary on the unit (e.g., subject) level. For example, there may be an effect on the survival time of a subject from that subject having taken some treatment (e.g., a fixed effect), and there may be additional effects on the survival time from unobserved genetic mutations. Unmeasured sources of variation, such as these unobserved genetic mutations, may be modeled in the subject-level random effects on subject survival. Such subject-level effects may also vary with measured features, but they may still take on different values across subjects.

In the limit of a large number of small unobserved additive effects, the distribution of random effects on a per-unit basis may tend to follow a normal distribution. Deviations from a normal distribution may be thus indicative that there may be underlying sources of variation in outcomes that may be clinically relevant (e.g., that they have effects comparable to or larger than other known sources of variation).

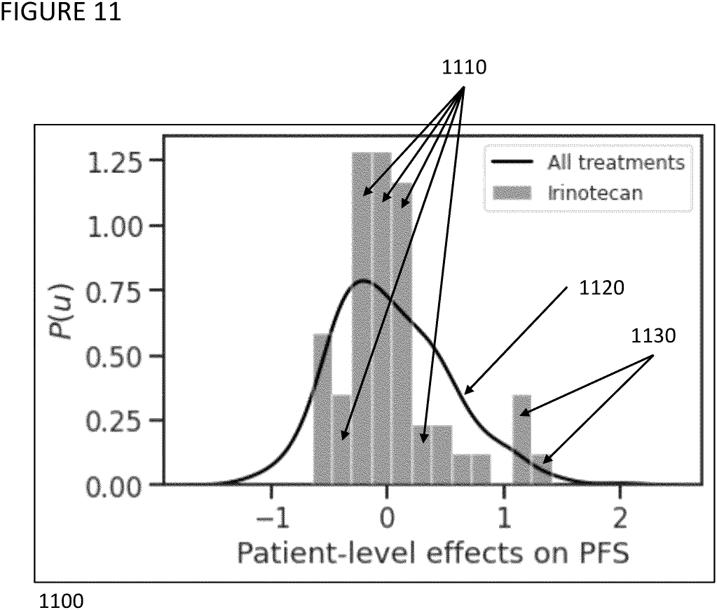

FIG. 11 shows the distribution of subject-level effects on progression-free survival (PFS) in an analysis of brain cancer subject data. Graph 1100 shows the distribution of subject-level effects for subjects treated with the systemic chemotherapy irinotecan, compared to all treatments for brain cancer. As may be seen in curve 1120, PFS may follow a roughly normal distribution. However, the distribution of subjects who were treated with the irinotecan may be significantly different from a normal distribution. The middle part of the irinotecan distribution may appear somewhat normal, as shown by bars 1110. However, there may be a cluster of subjects 1130 with better than expected outcomes.

By identifying clusters of subject-level random effects terms, it may be possible to classify subpopulations of interest to be examined in more detail to discover better predictors for likelihood of treatment response or survival time.

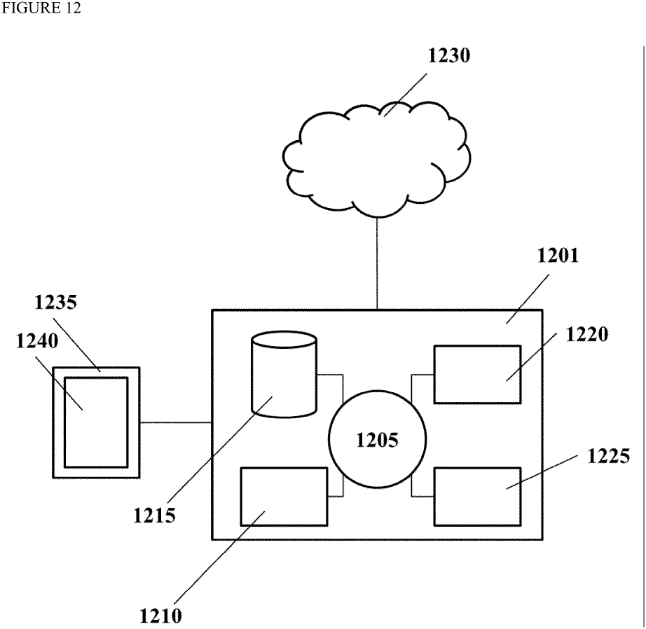

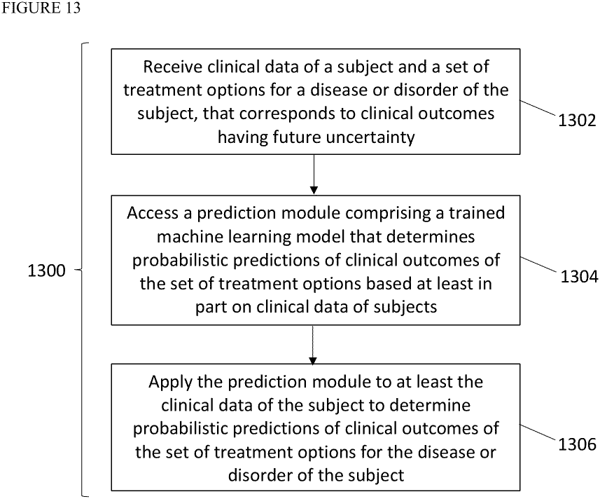

The present disclosure provides computer systems that may be programmed to implement methods of the disclosure. FIG. 12 shows a computer system 1201 that may be programmed or otherwise configured to, for example, (i) receive clinical data of a subject and a set of treatment options for a disease or disorder of the subject, (ii) access a prediction module comprising a trained machine learning model that determines probabilistic predictions of clinical outcomes of the set of treatment options based at least in part on clinical data of subjects, and (iii) apply the prediction module to clinical data of the subject, treatment features, and/or interaction terms to determine probabilistic predictions of clinical outcomes of the set of treatment options for the disease or disorder of the subject.

The computer system 1201 can regulate various aspects of analysis, calculation, and generation of the present disclosure, such as, for example, (i) receiving clinical data of a subject and a set of treatment options for a disease or disorder of the subject, (ii) accessing a prediction module comprising a trained machine learning model that determines probabilistic predictions of clinical outcomes of the set of treatment options based at least in part on clinical data of subjects, and (iii) applying the prediction module to clinical data of the subject, treatment features, and/or interaction terms to determine probabilistic predictions of clinical outcomes of the set of treatment options for the disease or disorder of the subject. The computer system 1201 can be an electronic device of a user or a computer system that may be remotely located with respect to the electronic device. The electronic device can be a mobile electronic device.

The computer system 1201 includes a central processing unit (CPU, also “processor” and “computer processor” herein) 1205, which can be a single core or multi core processor, or a plurality of processors for parallel processing. The computer system 1201 also includes memory or memory location 1210 (e.g., random-access memory, read-only memory, flash memory), electronic storage unit 1215 (e.g., hard disk), communication interface 1220 (e.g., network adapter) for communicating with one or more other systems, and peripheral devices 1225, such as cache, other memory, data storage and/or electronic display adapters. The memory 1210, storage unit 1215, interface 1220 and peripheral devices 1225 may be in communication with the CPU 1205 through a communication bus (solid lines), such as a motherboard. The storage unit 1215 can be a data storage unit (or data repository) for storing data. The computer system 1201 can be operatively coupled to a computer network (“network”) 1230 with the aid of the communication interface 1220. The network 1230 can be the Internet, an internet and/or extranet, or an intranet and/or extranet that may be in communication with the Internet.

The network 1230 in some cases may be a telecommunication and/or data network. The network 1230 can include one or more computer servers, which can enable distributed computing, such as cloud computing. For example, one or more computer servers may enable cloud computing over the network 1230 (“the cloud”) to perform various aspects of analysis, calculation, and generation of the present disclosure, such as, for example, (i) receiving clinical data of a subject and a set of treatment options for a disease or disorder of the subject, (ii) accessing a prediction module comprising a trained machine learning model that determines probabilistic predictions of clinical outcomes of the set of treatment options based at least in part on clinical data of subjects, and (iii) applying the prediction module to clinical data of the subject, treatment features, and/or interaction terms to determine probabilistic predictions of clinical outcomes of the set of treatment options for the disease or disorder of the subject. Such cloud computing may be provided by cloud computing platforms such as, for example, Amazon Web Services (AWS), Microsoft Azure, Google Cloud Platform, and IBM cloud. The network 1230, in some cases with the aid of the computer system 1201, can implement a peer-to-peer network, which may enable devices coupled to the computer system 1201 to behave as a client or a server.

The CPU 1205 may comprise one or more computer processors and/or one or more graphics processing units (GPUs). The CPU 1205 can execute a sequence of machine-readable instructions, which can be embodied in a program or software. The instructions may be stored in a memory location, such as the memory 1210. The instructions can be directed to the CPU 1205, which can subsequently program or otherwise configure the CPU 1205 to implement methods of the present disclosure. Examples of operations performed by the CPU 1205 can include fetch, decode, execute, and writeback.

The CPU 1205 can be part of a circuit, such as an integrated circuit. One or more other components of the system 1201 can be included in the circuit. In some cases, the circuit may be an application specific integrated circuit (ASIC).

The storage unit 1215 can store files, such as drivers, libraries and saved programs. The storage unit 1215 can store user data, e.g., user preferences and user programs. The computer system 1201 in some cases can include one or more additional data storage units that may be external to the computer system 1201, such as located on a remote server that may be in communication with the computer system 1201 through an intranet or the Internet.

The computer system 1201 can communicate with one or more remote computer systems through the network 1230. For instance, the computer system 1201 can communicate with a remote computer system of a user. Examples of remote computer systems include personal computers (e.g., portable PC), slate or tablet PC’s (e.g., Apple® iPad, Samsung® Galaxy Tab), telephones, Smart phones (e.g., Apple® iPhone, Android-enabled device, Blackberry®), or personal digital assistants. The user can access the computer system 1201 via the network 1230.

Methods as described herein can be implemented by way of machine (e.g., computer processor) executable code stored on an electronic storage location of the computer system 1201, such as, for example, on the memory 1210 or electronic storage unit 1215. The machine executable or machine readable code can be provided in the form of software. During use, the code can be executed by the processor 1205. In some cases, the code can be retrieved from the storage unit 1215 and stored on the memory 1210 for ready access by the processor 1205. In some situations, the electronic storage unit 1215 can be precluded, and machine-executable instructions may be stored on memory 1210.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1201, can be embodied in programming. Various aspects of the technology may be thought of as “products” or “articles of manufacture” typically in the form of machine (or processor) executable code and/or associated data that may be carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1201 can include or be in communication with an electronic display 1235 that comprises a user interface (UI) 1240 for providing, for example, (i) a visual display indicative of training and testing of a trained algorithm, (ii) a visual display of data indicative of a cancer status of a subject, (iii) a quantitative measure of a cancer status of a subject, (iv) an identification of a subject as having a cancer status, or (v) an electronic report indicative of the cancer status of the subject. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 1205. The algorithm can, for example, (i) receive clinical data of a subject and a set of treatment options for a disease or disorder of the subject, (ii) access a prediction module comprising a trained machine learning model that determines probabilistic predictions of clinical outcomes of the set of treatment options based at least in part on clinical data of subjects, and (iii) apply the prediction module to clinical data of the subject, treatment features, and/or interaction terms to determine probabilistic predictions of clinical outcomes of the set of treatment options for the disease or disorder of the subject.

### EXAMPLES

### Example 1

A system of the present disclosure may perform the following method for an automated identification of anomalous subject subpopulations:
1. Construct a multilevel model for the outcome or endpoint of interest (e.g., progression-free survival time) where the total effect on the outcome includes subject-level effects.
2. Using computational Bayesian algorithms, sample from the posterior probability distribution of the model parameters as conditioned on the outcomes data.
3. Construct subsamples of subjects by splitting the sample by measured covariates (e.g., treatments, biomarkers, and combinations thereof), with splits being made until some threshold of minimum number of subjects may be met.
4. For each subject subsample, estimate the deviation from normality in subject-level effects by performing normality tests (e.g., Shapiro-Wilks).
5. Rank the subject subsamples by the probability that they deviate from a normal distribution, providing visual tools so that users of the system may visually inspect the most anomalous subsamples for clustering of random effects.
6. Quantify the significance of clustering by applying Bayesian model section on the number of clusters with, e.g., a Gaussian mixture model on the subject-level effects distribution.
7. Once clusters of subject-level effects have been detected in various groups, doctors and scientists with domain-specific knowledge, or other users of a system or method of the present disclosure, may use this information to determine where to look for additional predictors that may reduce the variance of the subject-level random effects. This normally painstaking task may be made significantly easier with a system of the present disclosure.

### Example 2

FIG. 13 shows an example workflow of a method 1300. The method 1300 may comprise receiving clinical data of a subject and a set of treatment options for a disease or disorder of the subject (as in operation 1302). In some embodiments, the set of treatment options corresponds to clinical outcomes having future uncertainty. Next, the method 1300 may comprise access a prediction module comprising a trained machine learning model that determines probabilistic predictions of clinical outcomes of the set of treatment options based at least in part on clinical data of subjects (as in operation 1304). In some embodiments, the trained machine learning model is trained using a plurality of disparate data sources. Next, the method 1300 may comprise apply the prediction module to at least the clinical data of the subject to determine probabilistic predictions of clinical outcomes of the set of treatment options for the disease or disorder of the subject (as in operation 1306).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-75. (canceled)

76. A system comprising a computer processor and a storage device having instructions stored thereon that are operable, when executed by the computer processor, to cause the computer processor to:
(i) receive clinical data of a subject and a set of treatment options for a disease or disorder of the subject, wherein the set of treatment options corresponds to clinical outcomes having future uncertainty;
(ii) access a prediction module comprising a trained machine learning model that determines probabilistic predictions of clinical outcomes of the set of treatment options based at least in part on clinical data of test subjects; and
(iii) apply the prediction module to at least the clinical data of the subject to determine probabilistic predictions of clinical outcomes of the set of treatment options for the disease or disorder of the subject.

77. The system of claim 76, wherein the clinical data is selected from somatic genetic mutations, germline genetic mutations, mutational burden, protein levels, transcriptome levels, metabolite levels, tumor size or staging, clinical symptoms, laboratory test results, and clinical history.

78. The system of claim 76, wherein the disease or disorder comprises cancer.

79. The system of claim 76, wherein (iii) comprises applying the prediction module to at least treatment features of the set of treatment options or interaction terms between the clinical data of the subject and the treatment features of the set of treatment options, to determine the probabilistic predictions of the clinical outcomes of the set of treatment options.

80. The system of claim 76, wherein the clinical outcomes having future uncertainty comprise a change in tumor size, a change in patient functional status, a time-to-disease progression, a time-to-treatment failure, or a progression-free survival time.

81. The system of claim 76, wherein the probabilistic predictions of clinical outcomes of the set of treatment options comprise statistical distributions of the clinical outcomes of the set of treatment options.

82. The system of claim 76, wherein the probabilistic predictions of clinical outcomes of the set of treatment options are explainable based on performing a query of the probabilistic predictions.

83. The system of claim 76, wherein the instructions are operable, when executed by the computer processor, to cause the computer processor to further apply a training module that trains the trained machine learning model, wherein the training module updates the trained machine learning model using the probabilistic predictions of the clinical outcomes of the set of treatment options generated in (iii).

84. The system of claim 76, wherein the trained machine learning model is selected from the group consisting of a Bayesian model, a support vector machine (SVM), a linear regression, a logistic regression, a random forest, and a neural network.

85. The system of claim 76, wherein the trained machine learning model comprises a multilevel statistical model that accounts for variation at a plurality of distinct levels of analysis or correlation of subject-level effects across the plurality of distinct levels of analysis.

86. The system of claim 85, wherein the multilevel statistical model comprises a generalized linear model.

87. The system of claim 86, wherein the generalized linear model comprises use of the expression:
$$η = X ⋅ β + Z ⋅ u$$
wherein η is a linear response, X is a vector of predictors for treatment effects fixed across subjects, β is a vector of fixed effects, Z is a vector of predictors for subject-level treatment effects, and u is a vector of subject-level effects.

88. The system of claim 86, wherein the generalized linear model comprises use of the expression:
$$y = {g}^{- 1} \left(η\right)$$
wherein η is a linear response, g is an appropriately chosen link function from observed data to the linear response, and y is an outcome variable of interest.

89. The system of claim 76, wherein the instructions are operable, when executed by the computer processor, to cause the computer processor to further generate an electronic report comprising the probabilistic predictions of clinical outcomes of the set of treatment options, and wherein the electronic report is used to select a treatment option from among the set of treatment options based at least in part on the probabilistic predictions of clinical outcomes of the set of treatment options.

90. The system of claim 89, wherein the selected treatment option is administered to the subject, and wherein the prediction module is further applied to outcome data of the subject that is obtained subsequent to administering the selected treatment option to the subject, to determine updated probabilistic predictions of the clinical outcomes of the set of treatment options.

91. A computer-implemented method comprising:
(i) receiving clinical data of a subject and a set of treatment options for a disease or disorder of the subject, wherein the set of treatment options corresponds to clinical outcomes having future uncertainty;
(ii) accessing a prediction module comprising a trained machine learning model that determines probabilistic predictions of clinical outcomes of the set of treatment options based at least in part on clinical data of test subjects; and
(iii) applying the prediction module to at least the clinical data of the subject to determine probabilistic predictions of clinical outcomes of the set of treatment options for the disease or disorder of the subject.

92. The method of claim 91, wherein the clinical data is selected from somatic genetic mutations, germline genetic mutations, mutational burden, protein levels, transcriptome levels, metabolite levels, tumor size or staging, clinical symptoms, laboratory test results, and clinical history.

93. The method of claim 91, wherein the disease or disorder comprises cancer.

94. The method of claim 91, wherein (iii) comprises applying the prediction module to at least treatment features of the set of treatment options or interaction terms between the clinical data of the subject and the treatment features of the set of treatment options, to determine the probabilistic predictions of the clinical outcomes of the set of treatment options.

95. The method of claim 91, wherein the clinical outcomes having future uncertainty comprise a change in tumor size, a change in patient functional status, a time-to-disease progression, a time-to-treatment failure, overall survival, or progression-free survival.

96. The method of claim 91, wherein the probabilistic predictions of clinical outcomes of the set of treatment options comprise statistical distributions of the clinical outcomes of the set of treatment options.

97. The method of claim 91, wherein the probabilistic predictions of clinical outcomes of the set of treatment options are explainable based on performing a query of the probabilistic predictions.

98. The method of claim 91, further comprising applying a training module that trains the trained machine learning model, wherein the training module updates the trained machine learning model using the probabilistic predictions of the clinical outcomes of the set of treatment options generated in (iii).

99. The method of claim 91, wherein the trained machine learning model is selected from the group consisting of a Bayesian model, a support vector machine (SVM), a linear regression, a logistic regression, a random forest, and a neural network.

100. The method of claim 91, wherein the trained machine learning model comprises a multilevel statistical model that accounts for variation at a plurality of distinct levels of analysis or correlation of subject-level effects across the plurality of distinct levels of analysis.

101. The method of claim 100, wherein the multilevel statistical model comprises a generalized linear model.

102. The method of claim 101, wherein the generalized linear model comprises use of the expression:
$$η = X ⋅ β + Z ⋅ u$$
wherein η is a linear response, X is a vector of predictors for treatment effects fixed across subjects, β is a vector of fixed effects, Z is a vector of predictors for subject-level treatment effects, and u is a vector of subject-level effects.

103. The method of claim 101, wherein the generalized linear model comprises use of the expression:
$$y = {g}^{- 1} \left(η\right)$$
wherein η is a linear response, g is an appropriately chosen link function from observed data to the linear response, and y is an outcome variable of interest.

104. The method of claim 91, further comprising generating an electronic report comprising the probabilistic predictions of clinical outcomes of the set of treatment options, wherein the electronic report is used to select a treatment option from among the set of treatment options based at least in part on the probabilistic predictions of clinical outcomes of the set of treatment options.

105. The method of claim 104, wherein the selected treatment option is administered to the subject, and wherein the method further comprises applying the prediction module to outcome data of the subject that is obtained subsequent to administering the selected treatment option to the subject, to determine updated probabilistic predictions of the clinical outcomes of the set of treatment options.

106. A non-transitory computer storage medium storing instructions that are operable, when executed by computer processors, to cause the computer processor to implement a method comprising:
(i) receiving clinical data of a subject and a set of treatment options for a disease or disorder of the subject, wherein the set of treatment options corresponds to clinical outcomes having future uncertainty;
(ii) accessing a prediction module comprising a trained machine learning model that determines probabilistic predictions of clinical outcomes of the set of treatment options based at least in part on clinical data of test subjects; and
(iii) applying the prediction module to at least the clinical data of the subject to determine probabilistic predictions of clinical outcomes of the set of treatment options for the disease or disorder of the subject.
